(19) United States
(12) Patent Application Publication
(10) Pub. No.: US 2026/0119578 A1
(43) Pub. Date: Apr. 30, 2026

(54) SEMANTIC KNOWLEDGE GRAPH FOR CLINICAL SUMMARY GENERATION

(71) Applicant: Oracle International Corporation, Redwood Shores, CA (US)

(72) Inventors: Vadim Khotilovich, Leawood, KS (US); Andrew Roberts, Overland Park, KS (US)

(73) Assignee: Oracle International Corporation, Redwood Shores, CA (US)

(21) Appl. No.: 19/370,400

(22) Filed: Oct. 27, 2025

Related U.S. Application Data
(60) Provisional application No. 63/712,981, filed on Oct. 28, 2024.

(51) Int. Cl. G06F 16/901; G06F 16/28; G06N 3/042; G16H 15/00; G16H 50/70

(52) U.S. Cl. CPC G06F 16/9024; G06F 16/288; G06N 3/042; G16H 15/00; G16H 50/70

(57) ABSTRACT

Computer-implemented techniques are disclosed for constructing, augmenting, and utilizing graph-structured or other linked representations of data elements and associations derived from one or more sources to enable accurate, timely enrichment and analysis across multi-stage or other processing workflows. An intermediate representation of patient-specific data can be obtained. The intermediate representation can be processed to extract condition-related and medication-related information relevant to a patient encounter. Outputs can be processed to further filter and contextualize subsets of the condition-related and medication-related information. One or more filtering techniques including a knowledge-graph-based filtering technique can be applied. A clinical summary that includes facts derived from the intermediate representation can be generated.

### CROSS-REFENCE TO RELATED APPLICATION

This application claims priority to U.S. Provisional Application No. 63/712,981, filed on Oct. 28, 2025, the disclosure of which is incorporated herein by reference in its entirety for all purposes.

### FIELD

The present disclosure relates to computer-implemented techniques for constructing, augmenting, and utilizing graph-structured or other linked representations of data elements and associations derived from one or more sources to enable accurate, timely enrichment and analysis across multi-stage or other processing workflows.

### BACKGROUND

Computer-implemented systems that process heterogeneous data may employ one or more processing stages that, for example, identify and select task-relevant information, construct graph-structured or other linked representations of entities and relationships, and evaluate, infer, and curate relationship data for downstream use. Across such multi-stage workflows, gaps in source knowledge or representational structures, including missing nodes or edges, incomplete or unidirectional links, insufficient granularity, or uneven coverage, can cause relationship information pertinent to specific use cases to be omitted or underrepresented, reducing contextualization and degrading downstream prioritization and analysis. Accordingly, there remains a need for techniques that operate across multiple processing stages to expand recall and coverage over diverse concepts, surface relationships not explicitly present in source data, and provide accurate, low-latency outputs suitable for a range of data processing workflows.

### BRIEF SUMMARY

Techniques disclosed herein pertain to agentic artificial intelligence (AI) systems, and, more specifically, to computer-implemented techniques for constructing, augmenting, and utilizing graph-structured or other linked representations of data elements and associations derived from one or more sources to enable accurate, timely enrichment and analysis across multi-stage or other processing workflows.

In some embodiments, a computer-implemented method includes: obtaining, in response to a clinical query associated with an anticipated or ongoing patient encounter, an intermediate representation of patient-specific data, the intermediate representation comprising semantic objects extracted from one or more data sources, the semantic objects comprising a condition and a medication associated with a patient, and the intermediate representation further representing a reason for visit and a chief complaint for the encounter; processing the intermediate representation via a transform layer comprising multiple filtering modules to extract condition-related and medication-related information relevant to the encounter; processing outputs of the transform layer via an enrichment layer to further filter and contextualize subsets of the condition-related and medication-related information, wherein processing the outputs comprises extracting a subset of medications known to have therapeutic or adverse effects with respect to filtered conditions and extracting a subset of conditions affiliated with the subset of medications; within the enrichment layer, applying one or more filtering techniques including knowledge-graph-based filtering technique, wherein the knowledge-graph-based filtering technique prioritizes and retains entities according to a relevancy score; and generating, based on outputs of the transform layer and the enrichment layer, a clinical summary for the patient, the clinical summary comprising a narrative summary generated using natural language processing and a structured summary comprising structured facts derived from the intermediate representation.

In some embodiments, obtaining the intermediate representation comprises: receiving the clinical query; identifying, in the clinical query, clinical entities including at least medication entities and condition entities; and codifying the clinical entities using one or more coding systems to form a data structure compliant with an electronic healthcare information exchange standard.

In some embodiments, the method further includes: generating, using one or more knowledge sources and one or more large language models, a semantic knowledge graph representing clinical entities and typed relationships among the clinical entities; utilizing the semantic knowledge graph to prioritize and filter coded entities identified from the clinical query and the intermediate representation to produce link data comprising at least a prioritized condition list, a prioritized medication list, and relationships among conditions and medications; and supplying the link data to the enrichment layer to guide selection of clinically relevant entities for inclusion in the clinical summary.

In some embodiments, generating the semantic knowledge graph includes: executing a multi-stage pipeline including a discovery stage that applies a large language model to propose candidate relationships between clinical entities for which discretely documented links are absent in available knowledge sources; validating the candidate relationships; and persisting, for each candidate relationship of the candidate relationships, metadata comprising at least a source attribution that identifies a large language model, and a relationship type.

In some embodiments, the link data comprises relationship sets including: relationships that link a reason for visit or a chief complaint to candidate conditions relevant to encounter context; relationships that link the reason for visit or the chief complaint directly to candidate medications relevant to context of the encounter; relationships that link candidate conditions to candidate medications using relationship types; and relationships obtained indirectly by first identifying candidate conditions and then linking the candidate conditions to candidate medications.

In some embodiments, generating the clinical summary includes: populating a summary data structure with the link data such that a narrative portion of the clinical summary describes, in natural language, a context of the encounter and clinically prioritized entities and a structured portion of the clinical summary presents structured facts selected from the prioritized condition list, the prioritized medication list, and links that explain a rationale for prioritization and filtering.

In some embodiments, the method further includes: maintaining the semantic knowledge graph, wherein maintaining the semantic knowledge graph includes: normalizing source data to ontology-aligned representations; applying constraint and validation artifacts to detect incompleteness or inconsistency and reclassifying nodes or edges when ontologies or value sets are updated; and incrementally enriching the semantic knowledge graph over time by ingesting outputs of a large language model.

Some embodiments include a system that includes one or more processors and one or more computer-readable media storing instructions which, when executed by the one or more processors, cause the system to perform part or all of the operations and/or methods disclosed herein.

Some embodiments include one or more non-transitory computer-readable media storing instructions which, when executed by one or more processors, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 is a simplified block diagram of a distributed environment incorporating a digital assistant system in accordance with various embodiments.

FIG. 2 is an example of a user interface for configuring an agent of a digital assistant in accordance with various embodiments.

FIG. 3 is an example of a computing environment involving a digital assistant that can be implemented with generative artificial intelligence in accordance with various embodiments.

FIG. 4 is a simplified block diagram of a computing environment including a digital assistant that can execute an execution plan for responding to a query in accordance with various embodiments.

FIG. 5 is a simplified diagram of an example environment for providing a clinical artificial intelligence assistant, according to certain embodiments.

FIG. 6 is a simplified diagram of another example environment for providing a clinical artificial intelligence assistant, according to certain embodiments.

FIG. 7 is a simplified diagram of an example environment for providing a clinical digital assistant service, according to certain embodiments.

FIG. 8 is a simplified block diagram illustrating an example of a flow generating a clinical summary, according to certain embodiments.

FIG. 9 illustrates an example presentation format of summary semantic object, according to certain embodiments.

FIG. 10 is a simplified block diagram illustrating an example of a flow for generating and utilizing a semantic knowledge graph (SKG), according to certain embodiments.

FIG. 11 illustrates an example of entities that have been prioritized and filtered using the SKG, according to certain embodiments.

FIG. 12 illustrates an example of a data structure generated using an SKG, according to certain embodiments.

FIG. 13 illustrates another example of a data structure generated using an enhanced SKG, according to certain embodiments.

FIG. 14 illustrates an example of a pathway for utilizing an SKG, according to certain embodiments.

FIG. 15 illustrates another example of a pathway for utilizing an SKG, according to certain embodiments.

FIG. 16 illustrates another example of a pathway for utilizing an SKG, according to certain embodiments.

FIG. 17 depicts an example of a process for generating a clinical summary using an SKG, according to certain embodiments.

FIG. 18 depicts an example of a process for generating and maintaining an SKG, according to certain embodiments.

FIG. 19 is a block diagram illustrating one pattern for implementing a cloud infrastructure as a service system according to certain embodiments.

FIG. 20 is a block diagram illustrating another pattern for implementing a cloud infrastructure as a service system according to certain embodiments.

FIG. 21 is a block diagram illustrating another pattern for implementing a cloud infrastructure as a service system according to certain embodiments.

FIG. 22 is a block diagram illustrating another pattern for implementing a cloud infrastructure as a service system according to certain embodiments.

FIG. 23 is a block diagram illustrating an example computer system according to certain embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### Agentic Digital Assistant

Artificial intelligence techniques have broad applicability. For example, a digital assistant can be or include an artificial-intelligence-driven interface that helps users accomplish a variety of tasks using natural language conversations. For conventional digital assistants, such as those that do not involve generative artificial intelligence, a provider of the digital assistant may assemble one or more skills that can be focused on specific types of tasks such as tracking inventory, submitting timecards, and creating expense reports. When an end user engages with the digital assistant, the digital assistant can evaluate input provided by the end user to determine the intent of the end user and can route the conversation to and from the appropriate skill based on a perceived intent of the end user. However, there are some disadvantages of traditional intent-based skills including a limited understanding of natural language, inability to handle unknown inputs, limited ability to hold natural conversations off script, challenges integrating external knowledge, and the like.

User interactions with a digital assistant can lead to prompt responses to queries or the execution of requested actions. Additionally, these interactions have the potential to emulate human-like conversations, resembling a natural back-and-forth dialogue between a user and a human operator. To enhance user experience, digital assistants may also engage in multimodal communications, allowing users to convey information through spoken utterances or alternative input methods, such as selecting options on a computer display. However, achieving such functionalities efficiently with digital assistants, especially through natural language models, poses several challenges. For instance, understanding human speech remains a significant hurdle for natural language models, even those based on machine-learning. The scalability of the models can be problematic and inefficient, while their domain-specific limitations further complicate effective communication in various contexts.

The advent of generative artificial intelligence techniques and models, such as large language models (LLMs), has propelled the field of digital assistant design to unprecedented levels of sophistication and can be used to address the above and other technical problems associated with traditional intent-based skills. An LLM can be or include a neural network that employs a transformer architecture, which is specifically generated for processing and generating sequential data such as text or words in conversations. LLMs can undergo training with extensive textual data, and the training can gradually hone an ability to generate text that closely mimics human-written or spoken language.

Techniques are described herein to enhance LLMs with tools that empower or otherwise provide the LLMs access to external knowledge sources that provide the LLMs with the capability to recall facts and/or knowledge and facilitate the LLMs to better understand and respond to user queries in a contextually relevant manner. These tools, referred to herein as “agents,” provide the capability to recall facts and/or knowledge utilizing various techniques such as knowledge graphs, custom knowledge bases, Application Programming Interfaces (APIs), web crawling or scraping, and the like. In some examples, the tools, or the agents, can be powered or otherwise controlled by the LLMs. Once configured, the LLMs and agents can be deployed in artificial intelligence-based systems such as digital assistant applications. Users, such as end users or other entities, can interact with the digital assistant, such as by posing questions or making requests, and the LLMs and agents can work in tandem to generate responses based on a combination of a base LLM capability and access to the external knowledge via the agent. Using the LLMs and agents allows the digital assistant to provide more accurate, relevant, and contextually appropriate responses across a wide range of applications and domains.

For each digital assistant, a user (e.g., developer) may assemble LLMs and agents that interact to provide human-like conversation capabilities for various types of tasks such as tracking inventory, submitting timecards, updating accounts, creating expense reports, and the like. The LLMs are machine learning models trained on various tasks including plan creation using the LLM's in-context learning capability, knowledge or information retrieval on behalf of an agent, response generation to facilitate the human-like conversation, or any combination thereof. The agents are essentially containers having a software package containing everything needed to execute one or more actions defined for the agents. For example, the software package may include the code and any runtime configurations the code requires, application and system libraries, default values for any settings, and the like. The configuration parameters, settings, and customizations for dialog and routing/reasoning are primarily defined using natural language by a user (e.g., a developer). For example, users can provide configuration parameters that connect the agent to external assets, such as APIs, knowledge-based assets such as documents, URLs, LLMs, images, etc., data stores, prior conversations, etc. for executing one or more actions (e.g., change a user's 401k contribution). Once an agent is created, flow confirmation and testing may be performed through simulated conversations with LLMs and agents, and a digital assistant can then be implemented.

Implementation of an LLM-based digital assistant generally involves receiving a user input, such as a verbal request, command, or other statement (e.g., an utterance) from which the LLM digital assistant has a high-level awareness of the goal of the end user. A list of candidate agents is then determined based on the user input. The list of candidate agents includes agents configured to perform one or more actions that may potentially facilitate a response to the user input. Metadata for the agents in the list of candidate agents is then combined with the user input to construct an input prompt for an LLM. The LLM generates an execution plan that includes actions for facilitating a response to the user input based on the input prompt and metadata. The execution plan is then executed by an execution engine, which causes the agents to execute the actions. The actions may include internal task mapping in which a given action can be mapped to an API or semantic search knowledge task type. The execution of the actions generates output data from various sources, such as knowledge, API, SQL operations, etc., and/or relevant context and memory information from a context and memory store. The output data and relevant context and memory information are then combined with the user input to construct an output prompt for an LLM. The LLM synthesizes a response to the user input based on the output data and relevant context and memory information, and user input. The response is then sent to the user as an individual response or as part of a conversation with the user.

Advantageously, the LLM-based digital assistant described herein leverages reasoning capabilities of LLMs to drive decision-making and action orchestration to recall facts and/or knowledge and to facilitate the LLMs to better understand and respond to user queries in a contextually relevant manner. Additionally, or alternatively, the LLM-based digital assistant can eliminate a need for scripted dialog flows and provide out-of-the-box, human-like conversation capabilities.

A digital assistant can be or include a computer program that can perform conversations with end users. The digital assistant can generally respond to natural-language messages, such as questions and/or comments, through a messaging application (referred to herein as channels) that uses natural-language messages. The digital assistant can be made available to end users through a variety of channels, as well as via an application interface that may be developed to include a digital assistant, for example using a digital assistant software development kit. The channels may be or include an end-user-preferred messaging application that the end user has already installed and with which the end user may already be familiar. In some examples, the end user may not need to download and install new applications in order to converse with the digital assistant system. The channels may include, for example, over-the-top (OTT) messaging channels, such as Facebook™ Messenger, Facebook™ WhatsApp™, WeChat™, Line, Kik™, Telegram™, Talk, Skype™, Slack™, or SMS), virtual private assistants (such as Amazon™ Dot, Echo, or Show, Google™ Home, Apple™ HomePod™, etc.), mobile and web app extensions that extend native or hybrid/responsive mobile apps or web applications with chat capabilities, or voice based input such as devices or apps with interfaces that use Siri™, Cortana™, Google™ Voice, or other speech input for interaction.

The channels can carry the chat back and forth from end users to the digital assistant and various LLMs associated with the digital assistant. During the back-and-forth exchanges, the LLMs can receive the processed input in the form of a query and can process the query to generate a response. An LLM can predict the most contextually relevant and grammatically correct response based on training data used to train the LLM and based on received input such as the query and actions executed by the agents. The generated response may undergo post-processing to ensure adherence to guidelines, policies, and formatting standards associated with the digital assistant. This post-processed response may be more coherent and user-friendly than other responses that do not undergo post-processing. The post-processed response can be delivered to the user through the appropriate channel, which may be or include a text-based chat interface, a voice-based system, or another medium. According to various embodiments, the digital assistant can maintain the conversation context, allowing for further interactions and dynamic back-and-forth exchanges between the user and the LLMs where later interactions can build upon earlier interactions.

In some embodiments, the digital assistant system may intelligently handle end user interactions without interaction with a provider, such as an administrator or developer, of the digital assistant system. For example, an end user may send one or more messages to the digital assistant system in order to achieve a desired goal. A message may include certain content, such as text, emojis, audio, image, video, or other method of conveying a message. In some embodiments, the digital assistant system may convert the content into a standardized form, such as a representational state transfer (REST) or API call, against enterprise services with the proper parameters, and generate a natural language response. The digital assistant system may also prompt the end user for additional input parameters or request other additional information. In some embodiments, the digital assistant system may also initiate communication with the end user, rather than passively responding to end user utterances. Various techniques can be used for identifying an explicit invocation of a digital assistant system and determining an input for the digital assistant system being invoked. In certain embodiments, explicit invocation analysis can be performed by a primary digital assistant based at least in part on detecting an invocation name in an utterance. In response to detecting the invocation name, the utterance may be refined or pre-processed for input to a digital assistant that is identified to be associated with the invocation name and/or communication.

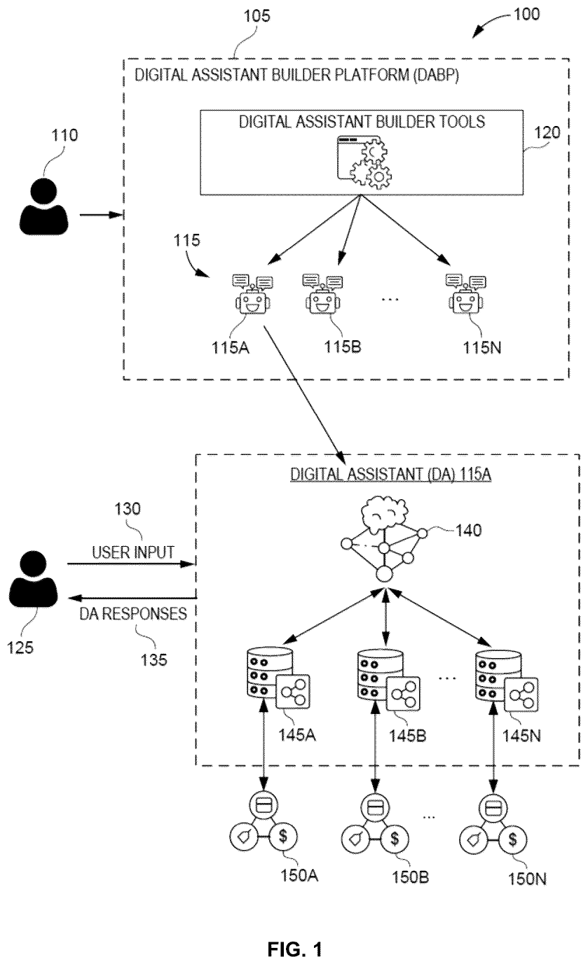

FIG. 1 is a simplified block diagram of an environment 100 incorporating a digital assistant system according to certain embodiments. Environment 100 includes a digital assistant builder platform (DABP) 105 that enables users 110 to create and deploy digital assistant systems 115. For purposes of this disclosure, a “digital assistant” is an entity that helps users of the digital assistant accomplish various tasks through natural language conversations. A digital assistant can be implemented using software only (e.g., the digital assistant is a digital entity implemented using programs, code, or instructions executable by one or more processors), using hardware, or using a combination of hardware and software. A digital assistant can be embodied or implemented in various physical systems or devices, such as in a computer, a mobile phone, a watch, an appliance, a vehicle, and the like. A digital assistant is also sometimes referred to as a chatbot system. Accordingly, for purposes of this disclosure, the terms digital assistant and chatbot system are interchangeable.

DABP 105 can be used to create one or more digital assistants (or DAs) systems. For example, as illustrated in FIG. 1, user 110 representing a particular enterprise can use DABP 105 to create and deploy a digital assistant 115A for users of the particular enterprise. For example, DABP 105 can be used by a bank to create one or more digital assistants for use by the bank's customers, for example to change a 401k contribution, etc. The same DABP 105 platform can be used by multiple enterprises to create digital assistants. As another example, an owner of a restaurant, such as a pizza shop, may use DABP 105 to create and deploy digital assistant 115B that enables customers of the restaurant to order food (e.g., order pizza).

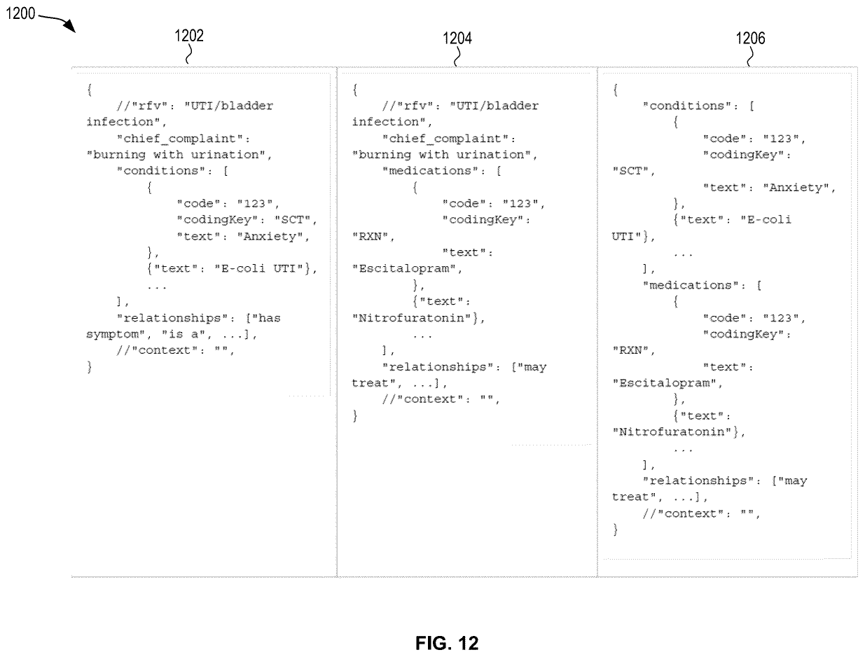

To create one or more digital assistant systems 115, the DABP 105 is equipped with a suite of tools 120, enabling the acquisition of LLMs, agent creation, asset identification, and deployment of digital assistant systems within a service architecture (described herein in detail with respect to FIG. 1) for users via a computing platform such as a cloud computing platform described in detail with respect to FIGS. 12-16. In some instances, the tools 120 can be utilized to access pre-trained and/or fine-tuned LLMs from data repositories or computing systems. The pre-trained LLMs serve as foundational elements, possessing extensive language understanding derived from vast datasets. This capability enables the models to generate coherent responses across various topics, facilitating transfer learning. Pre-trained models offer cost-effectiveness and flexibility, which allows for scalable improvements and continuous pre-training with new data, often establishing benchmarks in Natural Language Processing (NLP) tasks. Conversely, fine-tuned models are specifically trained for tasks or industries (e.g., plan creation utilizing the LLM's in-context learning capability, knowledge or information retrieval on behalf of an agent, response generation for human-like conversation, etc.), enhancing their performance on specific applications and enabling efficient learning from smaller, specialized datasets. Fine-tuning provides advantages such as task specialization, data efficiency, quicker training times, model customization, and resource efficiency. In some embodiments, fine-tuning may be particularly advantageous for niche applications and ongoing enhancement.

In other instances, the tools 120 can be utilized to pre-train and/or fine-tune the LLMs. The tools 120, or any subset thereof, may be standalone or part of a machine-learning operationalization framework, inclusive of hardware components like processors (e.g., CPU, GPU, TPU, FPGA, or any combination), memory, and storage. This framework operates software or computer program instructions (e.g., TensorFlow, PyTorch, Keras, etc.) to execute arithmetic, logic, input/output commands for training, validating, and deploying machine-learning models in a production environment. In certain instances, the tools 120 implement the training, validating, and deploying of the models using a cloud platform such as Oracle Cloud Infrastructure (OCI). Leveraging a cloud platform can make machine-learning more accessible, flexible, and cost-effective, which can facilitate faster model development and deployment for developers.

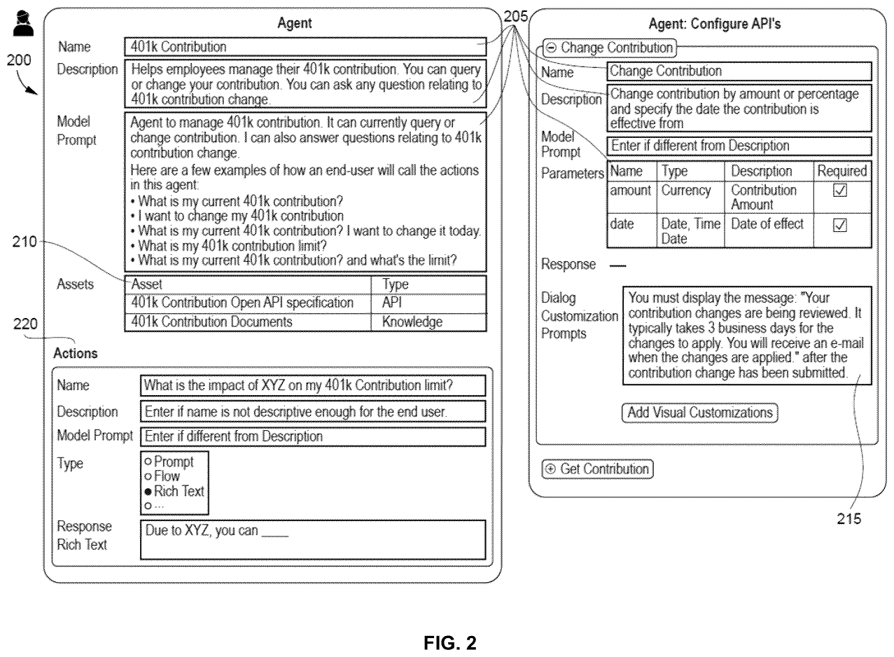

The tools 120 further include a prompt-based agent composition unit for creating agents and their associated actions (e.g., a prompt such as Tell me a joke, implicit Change Contribution, and Get Contribution API calls) that an end-user can end up invoking. As shown in FIG. 2, the agents (e.g., 401k Change Contribution Agent 200) are primarily defined as a compilation of agent artifacts 205 using natural language within the prompt-based agent composition unit. Users 110 can create functional agents quickly by providing agent artifact 205 information, parameters, and configurations and by pointing to assets 210. The assets 210 are resources such as APIs for interfacing with applications, files and/or documents for retrieving knowledge, data stores for interacting with data, and the like available to the agents for the execution of actions. The assets 210 are imported, and then the users 110 can use natural language again to provide additional API customizations for dialog and routing/reasoning 215. Most of what an agent does involves executing actions 220. An action 220 can be an explicit one that's authored using natural language (similar to creating agent artifacts—e.g., ‘What is the impact of XYZ on my 401k Contribution limit?’ action in the below ‘401k Contribution Agent’ figure) or an implicit one that is created when an asset is imported (automatically imported upon pointing to a given asset based on metadata and/or specifications associated with the asset—e.g., actions created for Change Contribution and Get Contribution API in the below ‘401k Contribution Agent’ figure). In the agent example illustrated in FIG. 2, the design time user can easily create explicit actions. For example, the user chooses the ‘Rich Text’ action type (see Table 1 for a list of exemplary action types) and creates the name artifact ‘What is the impact of XYZ on my 401k Contribution limit?’ when the user learns that a new FAQ needs to be added, as it's not currently in the knowledge documents (assets 210) the agent references (thus was not implicitly added as an action).

TABLE 1

| | Action Type | Description |
|---|---|---|
| 1 | Prompt | The action is implemented |
| | | using a prompt to an LLM. |
| 2 | Rich Text | The action is implemented using rich text. |
| | | The most common use case is |
| | | FAQs. |
| 3 | Flow | The action is implemented using |
| | | Visual Flow Designer flow. May be used |
| | | for complex cases where the developer |
| | | is not able to use the out-of-the-box |
| | | dialogue and dialog customizations. |

There are various ways in which the agents and assets can be associated or added to a digital assistant 115. In some instances, the agents can be developed by an enterprise and then added to a digital assistant using DABP 105. In other instances, the agents can be developed and created using DABP 105 and then added to a digital assistant created using DABP 105. In yet other instances, DABP 105 provides an online digital store (referred to as an “agent store”) that offers various pre-created agents directed to a wide range of tasks and actions. The agents offered through the agent store may also expose various cloud services. In order to add the agents to a digital assistant being generated using DABP 105, a user 110 of DABP 105 can access assets via tools 120, select specific assets for an agent, initiate a few mock chat conversations with the agent, and indicate that the agent is to be added to the digital assistant created using DABP 105.

Once deployed in a production environment, such as the architecture described with respect to FIG. 1, a digital assistant, such as digital assistant 115A built using DABP 105, can be used to perform various tasks via natural language-based conversations between the digital assistant 115A and its users 125. As described above, the digital assistant 115A illustrated in FIG. 1 can be made available or accessible to its users 125 through a variety of different channels, such as but not limited to, via certain applications, via social media platforms, via various messaging services and applications, and other applications or channels. A single digital assistant can have several channels configured for it so that it can be run on and be accessed by different services simultaneously.

As part of a conversation, a user 125 may provide one or more user inputs 130 to digital assistant 115A and get responses 135 back from digital assistant 115A. A conversation can include one or more user inputs 130 and responses 135. Via these conversations, a user 125 can request one or more tasks to be performed by the digital assistant 115A and, in response, the digital assistant 115A is configured to perform the user-requested tasks and respond with appropriate responses to the user 125 using one or more LLMs 140.

User inputs 130 are generally in a natural language form and are referred to as utterances, which may also be referred to as prompts, queries, requests, and the like. The user inputs 130 can be in text form, such as when a user types in a sentence, a question, a text fragment, or even a single word and provides it as input to digital assistant 115A. In some embodiments, a user input 130 can be in audio input or speech form, such as when a user says or speaks something that is provided as input to digital assistant 115A. The user inputs 130 are typically in a language spoken by the user 125. For example, the user inputs 130 may be in English, or some other language. When a user inputs 130 is in speech form, the speech input is converted to text form user inputs 130 in that particular language and the text utterances are then processed by digital assistant 115A. Various speech-to-text processing techniques may be used to convert a speech or audio input to a text utterance, which is then processed by digital assistant 115A. In some embodiments, the speech-to-text conversion may be done by digital assistant 115A itself. For purposes of this disclosure, it is assumed that the user inputs 130 are text utterances that have been provided directly by a user 125 of digital assistant 115A or are the results of conversion of input speech utterances to text form. This, however, is not intended to be limiting or restrictive in any manner.

The user inputs 130 can be used by the digital assistant 115A to determine a list of candidate agents 145A-N. The list of candidate agents (e.g., 145A-N) includes agents configured to perform one or more actions that could potentially facilitate a response 135 to the user input 130. The list may be determined by running a search, such as a semantic search, on a context and memory store that has one or more indices comprising metadata for all agents 145 available to the digital assistant 115A. Metadata for the candidate agents 145A-N in the list of candidate agents is then combined with the user input to construct an input prompt for the one or more LLMs 140.

Digital assistant 115A is configured to use one or more LLMs 140 to apply NLP techniques to text and/or speech to understand the input prompt and apply natural language understanding (NLU) including syntactic and semantic analysis of the text and/or speech to determine the meaning of the user inputs 130. Determining the meaning of the utterance may involve identifying the goal of the user, one or more intents of the user, the context surrounding various words or phrases or sentences, one or more entities corresponding to the utterance, and the like. The NLU processing can include parsing the received user inputs 130 to understand the structure and meaning of the utterance, refining and reforming the utterance to develop a better understandable form (e.g., logical form) or structure for the utterance. The NLU processing performed can include various NLP-related processing such as sentence parsing (e.g., tokenizing, lemmatizing, identifying part-of-speech tags for the sentence, identifying named entities in the sentence, generating dependency trees to represent the sentence structure, splitting a sentence into clauses, analyzing individual clauses, resolving anaphoras, performing chunking, and the like). In certain instances, the NLU processing, or any portions thereof, is performed by the LLMs 140 themselves. In other instances, the LLMs 140 use other resources to perform portions of the NLU processing. For example, the syntax and structure of an input utterance sentence may be identified by processing the sentence using a parser, a part-of-speech tagger, a named entity recognition model, a pretrained language model such as BERT, or the like.

Upon understanding the meaning of an utterance, the one or more LLMs 140 generate an execution plan that identifies one or more agents (e.g., agent 145A) from the list of candidate agents to execute and perform one or more actions or operations responsive to the understood meaning or goal of the user. The one or more actions or operations are then executed by the digital assistant 115A on one or more assets (e.g., asset 150A-knowledge, API, SQL operations, etc.) and/or the context and memory store. The execution of the one or more actions or operations generates output data from one or more assets and/or relevant context and memory information from a context and memory store comprising context for a present conversation with the digital assistant 115A. The output data and relevant context and memory information are then combined with the user input 130 to construct an output prompt for one or more LLMs 140. The LLMs 140 synthesize the response 135 to the user input 130 based on the output data and relevant context and memory information, and the user input 130. The response 135 is then sent to the user 125 as an individual response or as part of a conversation with the user 125.

For example, a user input 130 may request a pizza to be ordered by providing an utterance such as “I want to order a pizza.” Upon receiving such an utterance, digital assistant 115A is configured to understand the meaning or goal of the utterance and take appropriate actions. The appropriate actions may involve, for example, providing responses 135 to the user with questions requesting user input on the type of pizza the user desires to order, the size of the pizza, any toppings for the pizza, and the like. The questions requesting user may be generated by executing an action via an agent (e.g., agent 145A) on a knowledge asset (e.g., a menu for a pizza restaurant) to retrieve information that is pertinent to ordering a pizza (e.g., to order a pizza a user must provide type, seize, topping, etc.). The responses 135 provided by digital assistant 115A may also be in natural language form and typically in the same language as the user input 130. As part of generating these responses 135, digital assistant 115A may perform natural language generation (NLG) using the one or more LLMs 140. For the user ordering a pizza, via the conversation between the user and digital assistant 115A, the digital assistant 115A may guide the user to provide all the requisite information for the pizza order, and then at the end of the conversation cause the pizza to be ordered. The ordering may be performed by executing an action via an agent (e.g., agent 145A) on an API asset (e.g., an API for ordering pizza) to upload or provide the pizza order to the ordering system of the restaurant. Digital assistant 115A may end the conversation by generating a final response 135 providing information to the user 125 indicating that the pizza has been ordered.

While the various examples provided in this disclosure describe and/or illustrate utterances in the English language, this is meant only as an example. In certain embodiments, digital assistants 115 are also capable of handling utterances in languages other than English. Digital assistants 115 may provide subsystems (e.g., components implementing NLU functionality) that are configured for performing processing for different languages. These subsystems may be implemented as pluggable units that can be called using service calls from an NLU core server. This makes the NLU processing flexible and extensible for each language, including allowing different orders of processing. A language pack may be provided for individual languages, where a language pack can register a list of subsystems that can be served from the NLU core server.

While the embodiment in FIG. 1 illustrates the digital assistant 115A including one or more LLMs 140 and one or more agents 145A-N, this is not intended to be limiting. A digital assistant can include various other components (e.g., other systems and subsystems as described in greater detail with respect to FIG. 1) that provide the functionalities of the digital assistant. The digital assistant 115A and its systems and subsystems may be implemented only in software (e.g., code, instructions stored on a computer-readable medium and executable by one or more processors), in hardware only, or in implementations that use a combination of software and hardware.

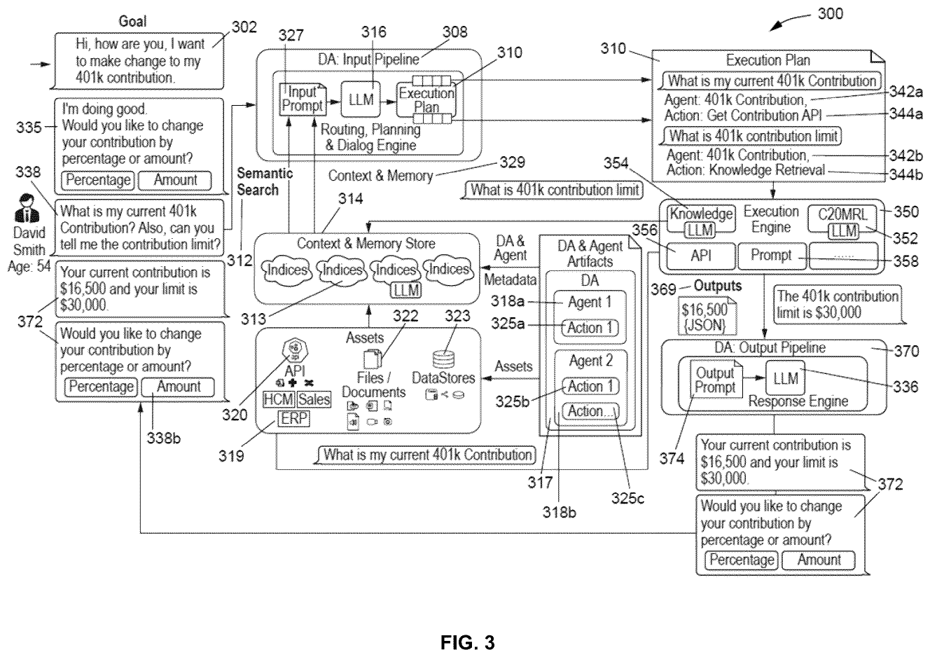

FIG. 3 is an example of an architecture for a computing environment 300 for a digital assistant implemented with generative artificial intelligence in accordance with various embodiments. As illustrated in FIG. 3, an infrastructure and various services and features can be used to enable a user to interact with a digital assistant (e.g., digital assistant 115A described with respect to FIG. 1) based at least in part on a series of prompts such as a conversation. The following is a detailed walkthrough of a conversation flow and the role and responsibility of the components, services, models, and the like of the computing environment 300 within the conversation flow. In this walkthrough, it is assumed that a user “David” is interested in making a change to his 401k contribution, and in an utterance 302, David provides the following input to the digital assistant: Hi, how are you, I want to make a change to my 401k contribution.

The utterance 302 can be communicated to the digital assistant (e.g., via text dialogue box or microphone) and provided as input to the input pipeline 308. The input pipeline 308 is used by the digital assistant to create an execution plan 310 that identifies one or more agents to address the request in the utterance 302 and one or more actions for the one or more agents to execute for responding to the request. A two-step approach can be taken via the input pipeline 308 to generate the execution plan 310. First, a search 312 can be performed to identify a list of candidate agents. The search 312 comprises running a query on indices 313 of a context and memory store 314 based on the utterance 302. In some instances, the search 312 is a semantic search performed using words from the utterance 302. The semantic search uses NLP and optionally machine learning techniques to understand the meaning of the utterance 302 and retrieve relevant information from the context and memory store 314. In contrast to traditional keyword-based searches, which rely on exact matches between the words in the query and the data in the context and memory store 314, a semantic search takes into account the relationships between words, the context of the query, synonyms, and other linguistic nuances. This allows the digital assistant to provide more accurate and contextually relevant results, making it more effective in understanding the user's intent in the utterance 302.

The context and memory store 314 is implemented using a data framework for connecting external data to LLMs 316 to make it easy for users to plug in custom data sources. The data framework provides rich and efficient retrieval mechanisms over data from various sources such as files, documents, datastores, APIs, and the like. The data can be external (e.g., enterprise assets) and/or internal (e.g., user preferences, memory, digital assistant, and agent metadata, etc.). In some instances, the data comprises metadata extracted from artifacts 317 associated with the digital assistant and its agents 318 (e.g., 318a and 318b). The artifacts 317 for the digital assistant include information on the general capabilities of the digital assistant and specific information concerning the capabilities of each of the agents 318 (e.g., actions 220 described with respect to FIG. 2) available to the digital assistant (e.g., agent artifacts 205 described with respect to FIG. 2). Additionally, or alternatively, the artifacts 317 can encompass parameters or information associated with the artifacts 317 and that can be used to define the agents 318 in which the parameters or information associated with the artifacts 317 can include a name, a description, one or more actions, one or more assets, one or more customizations, etc. In some instances, the data further includes metadata extracted from assets 319 associated with the digital assistant and its agents 318 (e.g., 318a and 318b). The assets 319 may be resources, such as APIs 320, files and/or documents 322, data stores 323, and the like, available to the agents 318 for the execution of actions (e.g., actions 325a, 325b, and 325c). The data is indexed in the context and memory store 314 as indices 313, which are data structures that provide a fast and efficient way to look up and retrieve specific data records within the data. Consequently, the context and memory store 314 provides a searchable comprehensive record of the capabilities of all agents and associated assets that are available to the digital assistant for responding to the request.

The results of the search 312 include a list of candidate agents that are not just available to the digital assistant for responding to the request but also potentially capable of facilitating the generation of a response to the utterance 302. The list of candidate agents includes the metadata (e.g., metadata extracted from artifacts 317 and assets 319) from the context and memory store 314 that is associated with each of the candidate agents. The list can be limited to a predetermined number of candidate agents (e.g., top 10) that satisfy the query or can include all agents that satisfy the query. The list of candidate agents with associated metadata is appended to the utterance 302 to construct an input prompt 327 for the LLM 316. In some instances, context 329 concerning the utterance 302 are additionally appended to the list of candidate agents and the utterance 302. The context 329 is retrievable from the context and memory store 314 and includes user session information, dialog state, conversation or contextual history, user information, or any combination thereof. The search 312 is important to the digital assistant because it filters out agents that are unlikely to be capable of facilitating the generation of a response to the utterance 302. This filter ensures that the number of tokens (e.g., word tokens) generated from the input prompt 327 remains under a maximum token limit or context limit set for the LLM 316. Token limits represent the maximum amount of text that can be inputted into an LLM. This limit is of a technical nature and arises due to computational constraints, such as memory and processing resources, and thus makes certain that the LLMs are capable of taking the input prompt as input.

The second step of the two-step approach is for the LLM 316 to generate an execution plan 310 based on the input prompt 327. The LLM 316 has a deep generative model architecture (e.g., a reversible or autoregressive architecture with) for generating the execution plan 310. In some instances, the LLM 316 has over 100 billion parameters and generates the execution plan 310 using autoregressive language modeling within a transformer architecture, allowing the LLM 316 to capture complex patterns and dependencies in the input prompt 327. The LLM's 316 ability to generate the execution plan 310 is a result of its training on diverse and extensive textual data, enabling the LLM to understand human language across a wide range of contexts. During training, the LLM 316 learns to predict the next word in a sequence given the context of the preceding words. This process involves adjusting the model's parameters (weights and biases) based on the errors between its predictions and the actual next words in the training data. When the LLM 316 receives an input such as the input prompt 327, the LLM 316 tokenizes the text into smaller units such as words or sub-words. Each token is then represented as a vector in a high-dimensional space. The LLM 316 processes the input sequence token by token, maintaining an internal representation of context. The LLM's 316 attention mechanism allows it to weigh the importance of different tokens in the context of generating the next word. For each token in the vocabulary, the LLM 316 calculates a probability distribution based on its learned parameters. This probability distribution represents the likelihood of each token being the next word given the context. To generate the execution plan 310, the LLM 316 samples a token from the calculated probability distribution. The sampled token becomes the next word in the generated sequence. This process is repeated iteratively, with each newly generated token influencing the context for generating the subsequent token. The LLM 316 can continue generating tokens until a predefined length or stopping condition is reached.

In some instances, as illustrated in FIG. 3, the LLM 316 may not be able to generate a complete execution plan 310 because it is missing information such as if more information is required to determine an appropriate agent for the response, execute one or more actions, or the like. In this particular instance, the LLM 316 has determined that in order to change the 401k contribution as request by the user, it is necessary to understand whether the user would like to change the contribution by a percentage or certain currency amount. In order to obtain this information, the LLM 316 (or another LLM such as LLM 336) generates end-user response 335 (I'm doing good. Would you like to change your contribution by percentage or amount? [Percentage] [Amount]) to the input prompt 327 that can obtain the missing information such that the LLM 316 is able to generate a complete execution plan 310. In some instances, the response may be rendered within a dialogue box of a GUI having one or more GUI elements allowing for an easier response by the user. In other instances, the response may be rendered within a dialogue box of a GUI allowing for the user to reply using the dialogue box (or alternative means such as a microphone). In this particular instance, the user responds with an additional query 338 (What is my current 401k Contribution? Also, can you tell me the contribution limit?) to gather additional information such that the user can reply to the response 335. The subsequent response-additional query 338—is input into the input pipeline 308 and the same processes described above with respect to utterance 302 are executed but this time with the context of the prior utterances/replies (e.g., utterance 302 and response 335) from the user's conversation with the digital assistant. This time, as illustrated in FIG. 3, the LLM 316 is able to generate a complete execution plan 310 because it has all the information it needs.

The execution plan 310 includes an ordered list of agents and/or actions that can be used and/or executed to sufficiently respond to the request such as the additional query 338. For example, and as illustrated in FIG. 3, the execution plan 310 can be an ordered list that includes a first agent 342a capable of executing a first action 344a via an associated asset and a second agent 342b capable of executing a second action 344b via an associated asset. The agents, and by extension the actions, may be ordered to cause the first action 344a to be executed by the first agent 342a prior to causing the second action 344b to be executed by the second agent 342b. In some instances, the execution plan 310 may be ordered based on dependencies indicated by the agents and/or actions included in the execution plan 310. For example, if executing the second agent 342b is dependent on, or otherwise requires, an output generated by the first agent 342a executing the first action 344a, then the execution plan 310 may order the first agent 342a and the second agent 342b to comply with the dependency. As should be understood, other examples of dependencies are possible.

The execution plan 310 is then transmitted to an execution engine 350 for implementation. The execution engine 350 includes a number of engines, including a natural language-to-programming language translator 352, a knowledge engine 354, an API engine 356, a prompt engine 358, and the like for executing the actions of agents and implementing the execution plan 310. For example, the natural language-to-programming language translator 352, such as a Conversation to Oracle Meaning Representation Language (C2OMRL) model, may be used by an agent to translate natural language into a intermedial logical for (e.g., OMRL), convert the intermediate logical form into a system programming language (e.g., SQL) and execute the system programming language (e.g., execute an SQL query) on an asset 319 such as data stores 323 to execute actions and/or obtain data or information. The knowledge engine 354 may be used by an agent to obtain data or information from the context and memory store 314 or an asset 319 such as files/documents 322. The API engine 356 may be used by an agent to call an API 320 and interface with an application such as retirement fund account management application to execute actions and/or obtain data or information. The prompt engine 358 may be used by an agent to construct a prompt for input into an LLM such as an LLM in the context and memory store 314 or an asset 319 to execute actions and/or obtain data or information.

The execution engine 350 implements the execution plan 310 by running each agent and executing each action in order based on the ordered list of agents and/or actions using the appropriate engine(s). To facilitate this implementation, the execution engine 350 is communicatively connected (e.g., via a public and/or provue network) with the agents (e.g., 342a, 342b, etc.), the context and memory store 314, and the assets 319. For example, as illustrated in FIG. 3, when the execution engine 350 implements the execution plan 310, it will first execute the agent 342a and action 344a using API engine 356 to call the API 320 and interface with a retirement fund account management application to retrieve the user's current 401k contribution. Subsequently, the execution engine 350 can execute the agent 342b and action 344b using knowledge engine 354 to retrieve knowledge on 401k contribution limits. In some instances, the knowledge is retrieved by knowledge engine 354 from the assets 319 (e.g., files/documents 322). In other instances (as in this particular instance), the knowledge is retrieved by knowledge engine 354 from the context and memory store 314. Knowledge retrieval and action execution using the context and memory store 314 may be implemented using various techniques including internal task mapping and/or machine learning models such as additional LLM models. For example, the query and associated agent for “What is 401k contribution limit” may be mapped to a ‘semantic search’ knowledge task type for searching the indices 313 within the context and memory store 314 for a response to a given query. By way of another example, a request such as “Can you summarize the key points relating to 401k contribution” can be or include a ‘summary’ knowledge task type that may be mapped to a different index within the context and memory store 314 having an LLM trained to create a natural language response (e.g., summary of key points relating to 401k contribution) to a given query. Over time, a library of generic end-user task or action types (e.g., semantic search, summarization, compare/contrast, heterogeneous data synthesis, etc.) may be built to ensure that the indices and models within the context and memory store 314 are optimized to the various task or action types.

The result of implementing the execution plan 310 is output data 369 (e.g., results of actions, data, information, etc.), which is transmitted to an output pipeline 370 (also referred to herein as response engine 370) for generating end-user responses 372. For example, the output data 369 from the assets 319 (knowledge, API, dialog history, etc.) and relevant information from the context and memory store 314 can be transmitted to the output pipeline 370. The output data 369 is appended to the utterance 302 to construct an output prompt 374 for input to the LLM 336. In some instances, context 329 concerning the utterance 302 are additionally appended to the output data 369 and the utterance 302. The context 329 is retrievable from the context and memory store 314 and includes user session information, dialog state, conversation or contextual history, user information, or any combination thereof. The LLM 336 generates responses 372 based on the output prompt 374. In some instances, the LLM 336 is the same or similar model as LLM 316. In other instances, the LLM 336 is different from LLM 316 (e.g., trained on a different set of data, a different architecture, trained for a one or more different tasks, etc.). In either instance, the LLM 336 has a deep generative model architecture (e.g., a reversible or autoregressive architecture with) for generating the responses 372 using similar training and generative processes described above with respect to LLM 316. In some instances, the LLM 336 has over 100 billion parameters and generates the responses 372 using autoregressive language modeling within a transformer architecture, allowing the LLM 336 to capture complex patterns and dependencies in the output prompt 374.

In some instances, the end-user responses 372 may be in the format of a Conversation Message Model (CMM) and output as rich multi-modal responses. The CMM defines the various message types that the digital assistant can send to the user (outbound), and the user can send to the digital assistant (inbound). In certain instances, the CMM identifies the following message types:
text: Basic text message
card: A card representation that contains a title and, optionally, a description, image, and link.
attachment: A message with a media URL (file, image, video, or audio)
location: A message with geo-location coordinates
postback: A message with a postback payload
   Messages that are defined in CMM are channel-agnostic and can be created using CMM syntax. The channel-specific connectors transform the CMM message into the format required by the specific channel, allowing a user to run the digital assistant on multiple channels without the need to create separate message formats for each channel.

Lastly, the output pipeline 370 transmits the responses 372 to the end user such as via a user device or interface. In some instances, the responses 372 are rendered within a dialogue box of a GUI allowing the user to view and reply using the dialogue box (or alternative means such as a microphone). In other instances, the responses 372 are rendered within a dialogue box of a GUI having one or more GUI elements allowing for an easier response by the user. In this particular instance, a first response 372 (What is my current 401k Contribution? Also, can you tell me the contribution limit?) to the additional query 338 is rendered within the dialogue box of a GUI. Additionally, in order to follow-up on obtaining information still required for the initial utterance 302, the LLM 336 generates another response 372 prompting the user for the missing information (Would you like to change your contribution by percentage or amount? [Percentage] [Amount]).

While the embodiment of computing environment 300 in FIG. 3 illustrates the digital assistant interacting in a particular conversation flow, this is not intended to be limiting and is merely provided to facilitate a better understanding of the role and responsibility of the components, services, models, and the like of the computing environment 300 within the conversation flow.

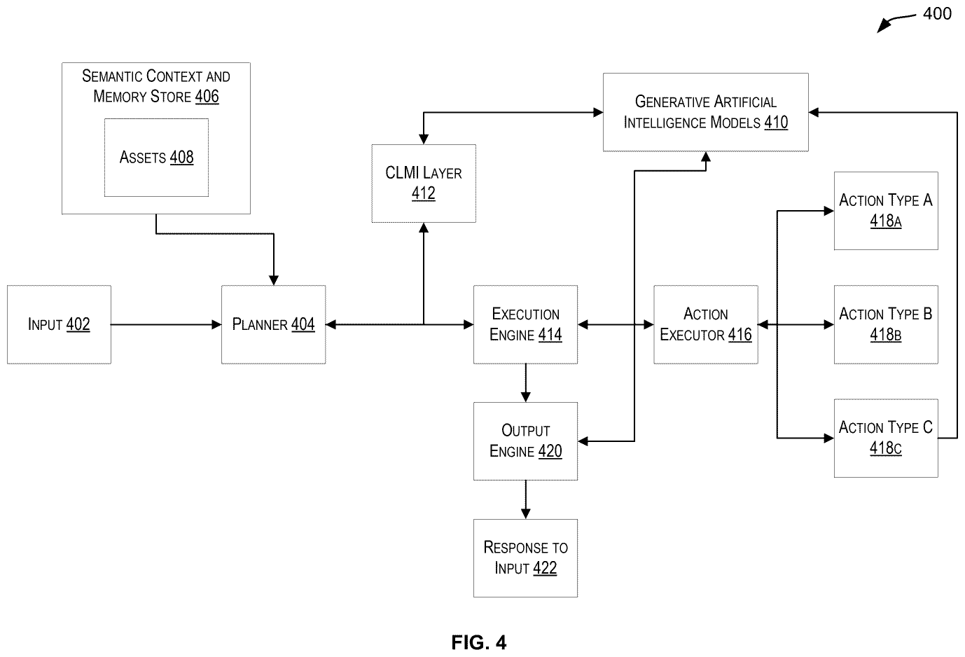

FIG. 4 is a simplified block diagram of a computing environment including a digital assistant 400 that can execute an execution plan for responding to an utterance from a user in accordance with various embodiments. In some embodiments, the utterance may be provided from the user to the digital assistant 400 via input 402. The input 402 may be or include natural language utterances that can include text input, voice input, image input, or any other suitable input for the digital assistant 400. For example, the input 402 may include text input provided by the user via a keyboard or touchscreen of a computing device used by the user. In other examples, the input 402 may include spoken words provided by the user via a microphone of the computing device. In other examples, the input 402 may include image data, video data, or other media provided by the user via the computing device. Additionally or alternatively, the input 402 may include indications of actions to be performed by the digital assistant 400 on behalf of the user. For example, the input 402 may include an indication that the user wants to order a pizza, that the user wants to update a retirement account contribution, or other suitable indications.

The input 402 may be provided to a planner 404 of the digital assistant 400. The planner 404 may generate an execution plan based on the input 402 and based on context provided to the planner 404. The planner 404 may receive the input 402 and may make a call to a semantic context and memory store 406 to retrieve the context. In some embodiments, the semantic context and memory store 406 includes one or more assets 408, which may be similar or identical to the assets 219. The planner 404 may provide at least a portion of the input 402 to the semantic context and memory store 406, which can perform a semantic search on the assets 408 and/or other knowledge included in the semantic context and memory store 406. The semantic search may generate a list of candidate actions, from among all actions that can be performed via one or more of the assets 408, that may be used to address the input 402 or any subset thereof. In some embodiments, the candidate actions may be generated only based on contextual information. For example, the input 402 may be compared with metadata of the actions to generate the candidate actions.

The planner 404 may use the candidate actions to form an input prompt for a generative artificial intelligence model. The generative artificial intelligence model may be or be included in generative artificial intelligence models 410, which may include one or more large language models (LLMs). The planner 404 may be communicatively coupled with the generative artificial intelligence models 410 via a common language model interface layer (CLMI layer 412). The CLMI layer 412 may be an adapter layer that can allow the planner 404 to call a variety of different generative artificial intelligence models that may be included in the generative artificial intelligence models 410. For example, the planner 404 may generate an input prompt and may provide the input prompt to the CLMI layer 412 that can convert the input prompt into a model-specific input prompt for being input into a particular generative artificial intelligence model. The planner 404 may receive output from the particular generative artificial intelligence model that can be used to generate an execution plan. The output may be or include the execution plan. In other embodiments, the output may be used as input by the planner 404 to allow the planner 404 to generate the execution plan. The output may include a list that includes one or more executable actions based on the utterance included in the input 402. In some embodiments, the execution plan may include an ordered list of actions to execute for addressing the input 402.

The planner 404 can transmit the execution plan to the execution engine 414 for executing the execution plan. The execution engine 414 may perform an iterative process for each executable action included in the execution plan. For example, the execution engine 414 may, for each executable action, identify an action type, may invoke one or more states for executing the action type, and may execute the executable action using an asset to obtain an output. The execution engine 414 may be communicatively coupled with an action executor 416 that may be configured to perform at least a portion of the iterative process. For example, the action executor 416 can identify one or more action types for each executable action included in the execution plan. In a particular example, the action executor 416 may identify a first action type 418a for a first executable action of the execution plan. The first action type 418a may be or include a semantic action such as summarizing text or other suitable semantic action. Additionally or alternatively, the action executor 416 may identify a second action type 418b for a second executable action of the execution plan. The second action type 418b may involve invoking an API such as an API for making an adjustment to an account or other suitable API. Additionally or alternatively, the action executor 416 may identify a third action type 418c for a third executable action of the execution plan. The third action type 418c may be or include a knowledge action such as providing an answer to a technical question or other suitable knowledge action. In some embodiments, the third action type 418c may involve making a call to at least one generative artificial intelligence model of the generative artificial intelligence models 410 to retrieve specific knowledge or a specific answer. In other embodiments, the third action type 418c may involve making a call to the semantic context and memory store 406 or other knowledge documents.

The action executor 416 may continue the iterative process based on the action types indicated by the executable actions included in the execution plan. Once the action executor 416 identifies the action types, the action executor 416 may identify and/or invoke one or more states for each executable action based on the action type. A state of an action may involve an indication of if or whether an action can be or has been executed. For example, the state for a particular executable action may include “preparing” “ready” “executing” “success” “failure” or any other suitable states. The action executor 416 can determine, based on the invoked state of the executable action, whether the executable action is ready to be executed, and, if the executable action is not ready to be execute, the action executor 416 can identify missing information or assets required for proceeding with executing the executable action. In response to determining that the executable action is ready to be executed, and in response to determining that no dependencies exist (or existing dependencies are satisfied) for the executable action, the action executor 416 can execute the executable action to generate an output.

The action executor 416 can execute each executable action, or any subset thereof, included in the execution plan to generate a set of outputs. The set of outputs may include knowledge outputs, semantic outputs, API outputs, and other suitable outputs. The action executor 416 may provide the set of outputs to an output engine 420. The output engine 420 may be configured to generate a second input prompt based on the set of outputs. The second input prompt can be provided to at least one generative artificial intelligence model of the generative artificial intelligence models 410 to generate a response 422 to the input 402. The output engine 420 may make a call to the at least one generative artificial intelligence model to cause the at least one generative artificial intelligence model to generate the response 422, which can be provided to the user in response to the input 402. In some embodiments, the at least one generative artificial intelligence model used to generate the response 422 may be similar or identical to, or otherwise the same model, as the at least one generative artificial intelligence model used to generate output for generating the execution plan.

As used herein, references to a LLM or LLMs are exemplary and non-limiting. The disclosed systems and methods are architecture-agnostic and apply to other model classes and sizes, including without limitation small language models (SLMs), large multimodel models (LMMs), multimodal large language models (MLLMs), vision-language models, speech-language models, encoder-only, decoder-only, and encoder-decoder transformers, convolutional and recurrent neural networks, graph neural networks, diffusion models, variational autoencoders (VAEs), generative adversarial networks (GANs), flow- or score-based models, retrieval-augmented models, ensembles, cascaded models, and hybrids thereof. Unless expressly stated otherwise, any functionality described with respect to an LLM may be implemented by any of the foregoing models and their equivalents.

As used herein, when an action is “based on” something, this means the action can be based at least in part on at least a part of the something. As used herein, the terms “similarly,” “substantially,” “approximately,” and “about” are defined as being largely but not necessarily wholly what is specified (and include wholly what is specified) as understood by one of ordinary skill in the art. In any disclosed embodiment, the term “similarly,” “substantially,” “approximately,” or “about” may be substituted with “within [a percentage] of” what is specified, where the percentage includes 0.1 percent, 1 percent, 5 percent, and 10 percent, etc.

### Clinical Artificial Intelligence Assistant (CAA)

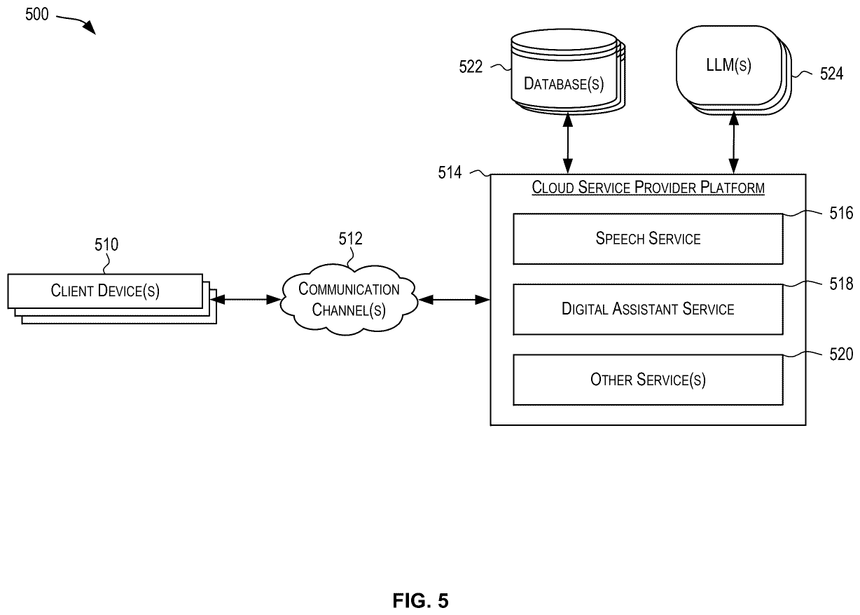

FIG. 5 shows a simplified diagram of an example environment 500 for providing a clinical artificial intelligence assistant (CAA). As shown in FIG. 5, the environment 500 includes one or more client devices 510 (hereinafter “client devices 510”), one or more communication channels 512 (hereinafter “communication channels”), a cloud service provider platform 514 (hereinafter “platform 514”), one or more databases 522 (hereinafter “databases 522”), and one or more LLMs 524 (hereinafter “LLMs”). The platform 514, which can be included as part of a cloud infrastructure of a cloud service provider (e.g., Oracle Cloud Infrastructure or OCI; the IaaS architecture 1200 of FIG. 12 described below), can be configured to communicate with, send data and information to, and receive data and information from the client devices 510 via the communication channels 512. Additionally, the platform 514 can be configured to access and/or call the databases 522 and the LLMs 524 to obtain and/or receive data and information from the databases 522 and the LLMs 524. Data and information received from the client devices 510, the databases 522, and the LLMs 524 can be used by the platform 514 to execute tasks and perform services such as a digital assistant service 518 that generates responses to a user query. While FIG. 5 shows the databases 522 and the LLMs 524 as being separate from the platform 514, this is not intended to be limiting, and one or more of the databases 522 and/or one or more of the LLMs 524 can be included as part of the platform 514 and/or the cloud infrastructure in which the platform 514 is included.

Each client device included in the client devices 510 can be any kind of electronic device that is capable of: executing applications; presenting information textually, graphically, and audibly such as via a display and a speaker; collecting information via one or more sensing elements such as image sensors, microphones, tactile sensors, touchscreen displays, and the like; connecting to a communication channel such as the communication channels 512 or a network such as a wireless network, wired network, a public network, a private network, and the like, to send and receive data and information; and/or storing data and information locally in one or more storage mediums of the electronic device and/or in one or more locations that are remote from the electronic device such as a cloud-based storage system, the platform 514, and/or the databases 522. Examples of electronic devices include, but are not limited to, mobile phones, desktop computers, portable computing devices, computers, workstations, laptop computers, tablet computers, and the like.

In some implementations, an application can be installed on, executing on, and/or accessed by a client device included in the client devices 510. The application and/or a user interface of the application can be utilized and/or interacted with (e.g., by an end user) to access, utilize, and/or interact with one or more services provided by the platform 514. The client device can be configured to receive multiple forms of input such as touch, text, voice, and the like, and the application can be configured to transform that input into one or more messages which can be transmitted or streamed to the platform 514 using one or more communication channels of the communication channels 512. Additionally, the client device can be configured to receive messages, data, and information from the platform 514 using one or more communication channels of the communication channels 512 and the application can be configured to present and/or render the received messages, data, and information in one or more user interfaces of the application.

Each communication channel included in the communication channels 512 can be any kind of communication channel that is capable of facilitating communication and the transfer of data and/or information between one or more entities such as the client devices 510, the platform 514, the databases 522, and the LLMs 524. Examples of communication channels include, but are not limited to, public networks, private networks, the Internet, wireless networks, wired networks, fiber optic networks, local area networks, wide area networks, and the like. The communication channels 512 can be configured to facilitate data and/or information streaming between and among the one or more entities. In some implementations, data and/or information can be streamed using one or more messages and according to one or more protocols. Each of the one or more messages can be a variable length message and each communication channel included in the communication channels 512 can include a stream orchestration layer that can receive the variable length message in accordance with a predefined interface, such as an interface defined using an interface description language like AsyncAPI. Each of the variable length messages can include context information that can be used to determine the route or routes for the variable length message as well as a text or binary payload of arbitrary length. Each of the routes can be configured using a polyglot stream orchestration language that is agnostic to the details of the underlying implementation of the routing tasks and destinations.

Each database included in the databases 522 can be any kind of database or knowledge base that is capable of storing data and/or information, providing access to data and/or information, and managing data and/or information. Data and/or information stored by each database can include data and/or information generated by, provided by, and/or otherwise obtained by the platform 514. Additionally, or alternatively, data and/or information stored and/or managed by each database can include data and/or information generated by, provided by, and/or otherwise obtained by other sources such as the client devices 510 and/or LLMs 524. One or more databases that are included in the databases 522 can be part of a platform for storing and managing healthcare information such as electronic health records for patients, electronic records of healthcare providers, and the like, and can store and manage electronic health records for patients of healthcare providers. An example platform is the Oracle Health Millenium Platform. Another example of a database that is included in the databases 522 is a semantic knowledge graph. Additionally, one or more databases included in the databases 522 can be provided by, managed by, and/or otherwise included as part of a cloud infrastructure of a cloud service provider (e.g., Oracle Cloud Infrastructure or OCI). Data and/or information stored and/or managed by the databases 522 can be accessed using one or more application programming interfaces (APIs) of the databases 522.

Each LLM included in the LLMs 524 can be any kind of LLM or generative machine learning model that is capable of obtaining or generating or retrieving one or more results in response to one or more inputs such as one or more prompts. Prompts for obtaining or generating or retrieving results from the LLMs 524 can obtained from or generated by or retrieved from or accessed from the client devices 510, the databases 512, the platform 514, the LLMs 524, and/or one or more other sources such as the Internet and other generative machine learning models. Each prompt can be configured to cause the LLMs 524 to perform one or more tasks, which causes one or more results to be provided or generated and the like. Prompts for the LLMs 524 can be pre-generated (i.e., before they are needed for a particular task) and/or generated in real-time (i.e., as they are needed for a particular task). In some implementations, prompts for the LLMs 524 can be engineered to achieve a desired result or results manually and/or by one or more machine-learning models. In some implementations, prompts for the LLMs 524 can be engineered one demand (i.e., in real-time and/or as needed) and/or at particular intervals (e.g., once per day, upon logging in by authenticated user into the platform 514). Each prompt of the one or more prompts can include a request for or a query for or a task to be performed by the LLMs 524 and contextual information. The contextual information can include information such as a text transcript or portions or segments thereof, information about an entity (e.g., information about a healthcare provider, information about a patient such as information included in an electronic health record for the patient, and the like), other information or records (e.g., lab results, ambient temperature, and the like), system instructions, candidate agent actions, and the like. LLMs included in the LLMs 524 can be pre-trained, fine-tuned, open source, off-the-shelf, licensed, subscribed to, and the like. Additionally, LLMs included in the LLMs 524 can include or have any size context window (i.e., can accept any number of tokens) and can be capable of interpreting complex instructions. One or more LLMs included in the LLMs 524 can be provided by, managed by, and/or otherwise included as part of the platform 514 and/or a cloud infrastructure of a cloud service provider (e.g., Oracle Cloud Infrastructure or OCI) that supports the platform 514. One or more LLMs included in the LLMs 524 can be accessed using one or more APIs of the LLMs 524 and/or a platform hosting or supporting or providing the LLMs 524.

The platform 514 can be configured to include various capabilities and provide various services to subscribers (e.g., end users) of the various services. In some implementations, in the case of an end user or subscriber being a healthcare provider, the healthcare provider can utilize the various services to facilitate the observation, care, treatment, management, and so on of their patient populations. For example, a healthcare provider can utilize the functionality provided by the various services provided by the platform 514 to examine and/or treat and/or facilitate the examination and/or treatment of a patient; view, edit, and/or manage a patient's electronic health record; perform administrative tasks such as scheduling appointments, managing patient populations, providing customer service to facilitate operation of a healthcare environment in which the healthcare provider practices, and so on.

In some implementations, the services provided by the platform 514 can include, but are not limited to, a speech service 516, the digital assistant service 518, and other service(s) 520 such as a SOAP Note service. The speech service 516 can be configured to convert audio such as a conversation into text such as a text transcript. For example, the speech service 516 can convert an audio recording of a conversation between a healthcare provider and a patient into a text transcript of the conversation. To convert audio into text, the speech service 516 can utilize one or more speech recognition techniques. For example, the speech service 516 can utilize a machine-learning model such as an automatic speech recognition (ASR) model. In the case that the audio is streamed to the platform 514 in the form of messages (as described above) with each message including a portion of the audio (e.g., a one second segment of the audio), in some implementations, the platform 514 and/or the speech service 516 can be configured to aggregate and combine all of the messages pertaining to the audio (e.g., all of the messages pertaining to a conversation) into audio data and/or an audio file prior to converting the audio data or audio file into text and/or a text transcript. In other implementations, the platform 514 and/or the speech service 516 can be configured to convert audio into text or a text transcript as the audio is received by the platform 514 and/or the speech service 516. The text or text transcript generated by the speech service 516 can be stored within the platform 514 and/or in another location such as in one or more databases of the databases 522, where it can be accessed by the platform 514, one or more other services of the platform 514 such as the digital assistant service 518 and/or SOAP note service 520, and/or the LLMs 524. Additionally, or alternatively, the text or text transcript generated by the speech service 516 can be provided to the digital assistant service 518 and/or the other service(s) 520, and/or the LLMs 524.

The digital assistant service 518 can be configured to serve as an artificial intelligence-driven (AI-driven) conversational-type interface for the platform 514 that can conduct conversations with end users (e.g., those using the client devices 510) and perform functions and/or tasks based on the information conveyed by and/or ascertained from those conversations and other sources. The digital assistant service 518 can be configured with and/or configured to access natural language understanding (NLU) capabilities such as natural language processing, named entity recognition, intent classification, and so on. In some implementations, the digital assistant service 518 can be LLM-based and agent-driven in which agent action(s) coordinate with LLM(s) for conducting conversations and performing functions and/or tasks such as the agentic digital assistant described above with respect to FIGS. 1-4.

The digital assistant service 518 can be configured to initiate a dialog, drive a previously initiated dialog (e.g., by responding to a turn in the dialog), and/or otherwise participate in a conversation. In some implementations, the digital assistant service 518 can drive and/or participate in a dialog and/or conversation in response to events that have occurred at the client devices 510, the platform 514, the databases 522, the LLMs 524, and/or at the cloud infrastructure supporting the platform 514. In the case of an LLM-based and agent-driven digital assistant service 518, events can be mapped to a particular prompt or prompts to retrieve a result or results for the prompt or prompts, which can then be used to render the user interface. In some implementations, the digital assistant service 518 can drive and/or participate in a dialog and/or conversation in response to messages received from the client devices 510, the platform 514, the databases 522, the LLMs 524, and/or at the cloud infrastructure supporting the platform 514. In the case of an LLM-based and agent-driven digital assistant service 18, the metadata included in the messages can be used to generate and/or access a particular prompt or prompts to retrieve a result and/or results that can be used to render the user interface.

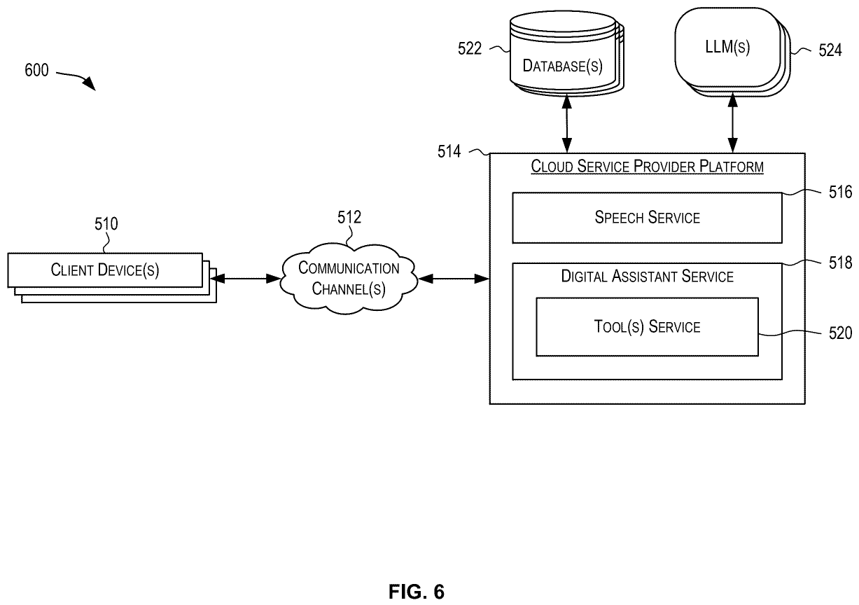

Although not shown, the platform 514 can include other capabilities and services such as authentication services, management services, task management services, notification services, and the like. The various capabilities and services of the platform 514 can be implemented utilizing one or more computing resources and/or servers of the platform 514 and provided by the platform 514 by way of subscriptions. Additionally, or alternatively, while FIG. 5 shows the services of the platform 514 as being separate services, one or more of the services can be combined with other services and/or be considered to be a sub-service of another service. For example, as shown in FIG. 6, in the environment 600, the other service(s) 520 can provide a sub-service of or be part of the digital assistant service 518.

The environments 500 and 600 depicted in FIGS. 5 and 6 are merely exemplary and are not intended to unduly limit the scope of claimed embodiments. One of ordinary skill in the art would recognize many possible variations, alternatives, and modifications. For example, in some implementations, the environments 500 and 600 can be implemented using more or fewer services than those shown in FIG. 5 and FIG. 6, may combine two or more services, or may have a different configuration or arrangement of services.

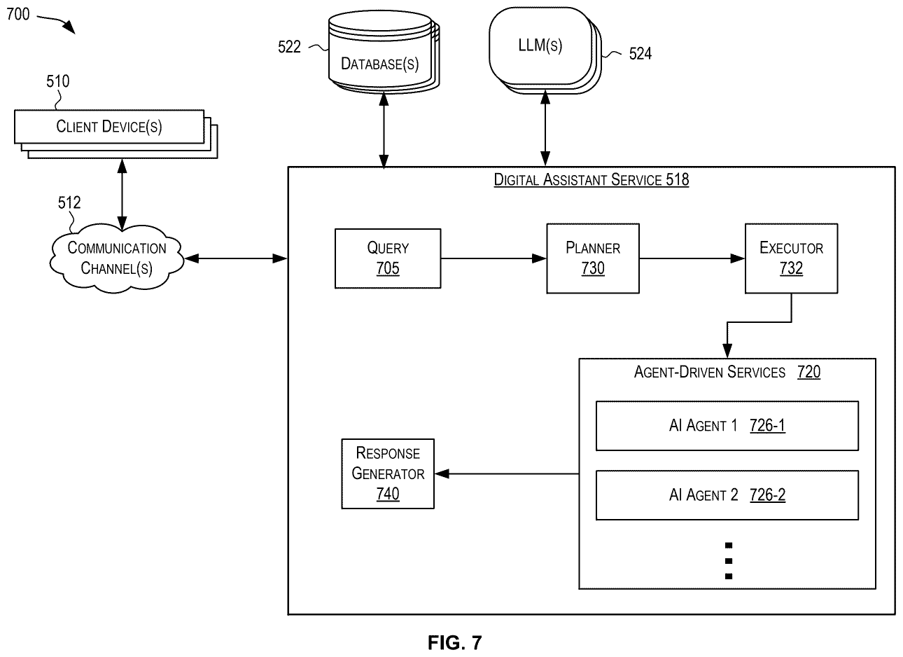

FIG. 7 is a simplified diagram of an example environment 700 for providing a clinical digital assistant service. The clinical digital assistant service can implement agent-driven services in which AI agents perform a defined task or set of tasks (e.g., a generating a clinical note or summary). In some implementations, the environment 700 includes the client devices 510, the communication channels 512, and the digital assistant service 518 of a cloud service provider platform 514. The digital assistant service 518 can include agent-driven services 720, which can include one or more AI agents such as AI Agent 1 726-1 and AI Agent 2 726-2. The digital assistant service 518 can utilize the AI agents to perform the defined task or set of tasks. Although two AI agents are shown, this is not intended to be limiting, and agent-driven services 720 can include any number of AI agents. Each AI agent can be configured to perform a particular task, such as the clinical summary generation disclosed herein. In some implementations, to perform a task, an AI Agent can call one or more LLMs such as an LLM of LLMs 124 and/or an LLM within the AI agent itself to generate an execution plan comprising a set of instructions for performing the task and then execute the execution plan to perform the task (e.g., retrieve data from a plurality of sources such as databases 522 and process the data to generate refined data to be used to generate a clinical summary).

In some implementations, digital assistant service 518 is configured to access a query 705 (e.g., a query received from the client devices 510 via communication channels 512) and provide the query 705 to a planner 730. The planner 730 generates an execution plan to perform the defined task or set of tasks. The execution plan can identify an AI agent or AI agents of the agent-driven services 720 that should be used to perform the defined task or set of tasks and an order (e.g., parallel, sequential) in which each AI agent should be executed. The planner 730 provides the execution plan to the executor 732, which can call each AI agent of the AI agents identified in the execution plan according to the order identified in execution plan. In some implementations, as described above, calling an AI agent causes the AI agent to perform a task or sub-task of the defined task or set of tasks. Performing a task or sub-task of the defined task or set of tasks results in the generation or retrieval of information associated with the task or the sub-task. The information generated/retrieved by each AI agent can be assembled and provided to the response generator 740 by the agent-driven services 720. The response generator 740 then uses the information to generate a response and a message incorporating the response, which can then be provided by the digital assistant service 518 to the client devices 510 for presentation a response to the query 705.

While aspects of the present disclosure are primarily described with reference to implementations utilizing large language models (LLMs), the described systems, methods, and functionalities are not limited to LLM-based architectures. Other generative machine learning techniques and models, both currently known and later developed, may be employed to accomplish the same or similar objectives described herein. Examples of generative machine learning models include, without limitation: small language models (SLMs) having fewer parameters relative to LLMs; multimodal generative models configured to process or generate two or more modalities (for example, text, image, audio, or video); generative natural language processing (NLP) models such as autoregressive language models or encoder-decoder sequence-to-sequence models used to produce natural-language outputs; transformer-based generative models, including decoder-only or encoder-decoder transformer architectures configured for generative inference; diffusion models configured to synthesize content via iterative denoising; variational autoencoders (VAEs); and generative adversarial networks (GANs). References to particular model families are exemplary and do not preclude the use of alternative generative models, frameworks, or architectures capable of generating, synthesizing, or manipulating digital content, responses, or actions in agentic AI systems and digital assistants.

### Clinical Summaries

Healthcare providers often find it useful to locate and review a variety of information regarding a patient prior to an encounter with the patient. Often, healthcare providers locate and review this information when assuming responsibility for a patient from another healthcare provider. For example, for patients admitted in a hospital setting, the care team typically transfers information about patients at shift overlaps and during handoffs between different members of the care team. Such clinical handoffs may occur several times a day during a patient's hospital stay. As a result, efficient retrieval of patient-specific information between healthcare providers and accurate knowledge transfer between healthcare providers is important to providing high quality patient care. However, locating, reviewing, and assembling the appropriate information for these handoffs can be difficult even with the proliferation of electronically accessible EHR systems. In many cases, to provide information, healthcare providers often obtain information from disparate sources including EHR systems and spend time reviewing, organizing, and assembling the information such that it will be useful.

Electronic and computerized tools have been developed and utilized by healthcare providers to perform these tasks, but these tools often lack the computational resources to perform these tasks with low latency and high accuracy. One challenge often encountered by these tools is the different coding schemes employed by different information storage sources. For example, many EHR systems use proprietary coding systems for storing patient information. Additionally, these tools often lack the capabilities to generate customized information based on patient status and/or healthcare provider status (e.g., a patient new to healthcare provider, a newly admitted patient, a new healthcare provider for the patient).

In many cases, intelligent tools such as agentic digital assistants have been employed to perform these tasks. These agentic digital assistants often utilize one or more generative machine learning models such as large language models (LLMs) to retrieve information related to an inquiry, process the information, and generate a response to the inquiry from the processed information. While these agentic digital assistants have been useful in improving information retrieval and synthesis, utilizing these assistants in clinical settings presents challenges. For example, EHRs often encompass extensive and fragmented information, including personal information, patient histories, test results, physician notes, and medication records stored using different coding schemes, although processing this vast context efficiently poses a significant challenge for a variety of reasons such as information overload, model limitations, temporal context, and patient-specific context. In another example, EHR data is rarely presented in a unified format with both structured fields (e.g., lab results, medication lists) and unstructured text (e.g., physician notes, patient complaints), and processing these different formats poses data fusion and aggregation challenges, semantic alignment challenges, and inconsistencies across healthcare providers. In yet another example, LLMs and other generative machine learning models are often pre-trained on general concepts, yet lack a deep understanding of clinical contexts, guidelines, textbooks, publications, ontologies, and medical reasoning, which often result in inaccuracies and can have severe consequences such as misdiagnosis and/or inappropriate treatments.

To address these challenges and others, automatic clinical summary generation techniques have been developed. The techniques described herein also provide a succinct clinical contextual summary presenting the patient's needs and status in a focused, curated manner with narrative and discrete details. The summaries include narrative and discrete detail to enable the recipient to quickly understand a given patient's status, with additional information readily accessible as needed. The summary can include what happened since the last time a physician cared for a patient (i.e. a summary of things that have changed) and/or a summary of what happened since the patient was admitted. Given a query received from a healthcare provider, EHR data and other data related to the patient is processed using a set of processing modules. A narrative summary then generated and a structured summary to provide the healthcare provider with a summary of clinical information specific to the patient.

It is noted that, the term “healthcare provider” as used herein generally refers to healthcare practitioners and professionals including, and not limited to: physicians (e.g., general practitioners, specialists, surgeons, etc.); nurse professionals (e.g., nurse practitioners, physician assistants, nursing staff, registered nurses, licensed practical nurses, etc.); and other professionals (e.g., pharmacists, therapists, technicians, technologists, pathologists, dietitians, nutritionists, emergency medical technicians, psychiatrists, psychologists, counselors, dentists, orthodontists, hygienists, etc.).

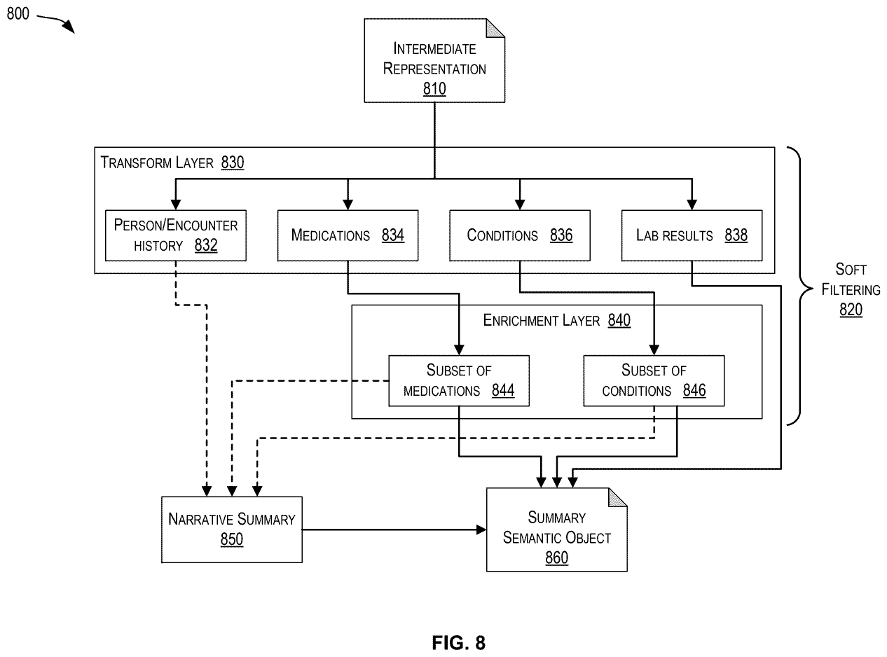

FIG. 8 is a simplified block diagram illustrating an example of a flow 800 for generating a clinical summary. As shown in FIG. 8, the flow 800 begins with an intermediate representation (IR) 810 of the relevant data, with the IR having a structured, computer-interpretable encoding format. The IR may be produced, for example, by extracting data (e.g., known facts, notes, etc.) specific to the patient from one or more databases, such as an EHR system, medical databases, other business records accessible to the healthcare provider, public resources, and others. The IR may also take into consideration a priori knowledge regarding the recorded chief complaint for an anticipated patient encounter, contextual information, and the like. The use of semantic objects from an EHR system as the source of IR data may be particularly advantageous as the semantic objects (SO) align closely with Fast Healthcare Interoperability Resources (FHIR) standards, thus providing ready adaptability to multiple EHR systems and other data resources following the FHIR standard. Examples of SOs include, but are not limited to: conditions, medications, labs, vitals, diagnostic reports, clinical documents, encounters, and allergies.

Examples of patient information that may be represented by the IR 810 include, but are not limited to: patient's name, age, and gender; patient's reason for visit (RFV); their chief complaint (CC); their last visit date and type; related visit notes; new related problems; new related medications; current and new allergies; new related diagnostic study data and lab results; new related procedures; related social history; related family history; current vitals; current nurse notes; and recommendations; future related appointments

The IR 810 is directed through a sequence of soft filtering layers 820. In some implementations, the soft filtering layers 820 include a transform layer 830 and an enrichment layer 840. In the transform layer 830, aspects of IR 810 can be filtered for commonly relevant facts. For example, the transform layer 230 can include a person/encounter history module 832 that is configured to filter person/encounter information from the IR 810 that may be relevant for the current visit. In another example, the transform layer 830 can include a medications module 834 that is configured to filter medications from the IR 810 that may be relevant for the current visit. In a further example, the transform layer 830 can include a conditions module 836 that is configured to filter conditions from the IR 810 that may be relevant for the current visit. In yet another example, the transform layer 830 can include a lab results module 838 that is configured to filter lab results from the IR 810 that may be relevant for the current visit.

In the enrichment layer 840, filter information provided by the medications module 834 and the conditions module 836 can be further filtered using a subset of medications module 844 and a subset of conditions module 846. For example, the medication-related information provided by the medications module 834 may be further gleaned for relevant data given the output from the conditions module 836 to extract a subset of medications known to have a therapeutic or adverse effect on known conditions. In another example, a subset of conditions may be extracted to highlight conditions known to be affiliated with the extracted medications and other a priori information extracted in transform layer 830.

In some implementations, the enrichment layer 240 can employ rule-based filtering techniques, knowledge graph-based filtering techniques, LLM-based filtering techniques, lexical matching-based filtering techniques, or a combination thereof. The rule-based filtering can apply targeted rules to the medication information provided by the medications module 834 (e.g., filtering the medications to a particular time window). The knowledge graph-based filtering can extract medication information and condition information that is related to the medication information and conditions information provided by the medications module 834 and the conditions module 836. In some implementations, the medication and condition information is extracted from the knowledge graph if a relevancy score between the extracted information the filtered information exceeds a predetermined threshold. The LLM-based filtering can use a LLM to predict medications that are related to the medications of the medication information provided by the medications module 834 and conditions that are related to the conditions of the condition information provided by the conditions module 836. In some implementations, the LLM can be instructed to systematically evaluate a predefined set of relationships before determining whether predicted entities are medically relevant given the medication and condition information. The lexical matching-based filtering can search clinical notes to identify clinical notes that are relevant the medication and condition information. In some implementations, notes that have the most occurrences of relevant entities for the target condition or medication are highly scored and retained.

The extracted portions of data from transform layer 830 and enrichment layer 840 may be used in crafting a narrative summary 850. For example, extracted IR data (with an example flow of data indicated by dashed arrows) may be compiled through natural language processing methods to generate narrative summary 850 based on the extracted information. In examples, narrative summary provides a pithy yet informative summary of patient information, particularly highlighting patient and encounter history as well as subset of medications and conditions relevant to the patient encounter. Narrative summary 850 may be provided as a standalone report and/or combined with other facts extracted from IR 810, depending on the summary type and use case, to generate a summary semantic object 860. For instance, in addition to a narrative summary section, structured data may also be used to populate a structured portion of summary semantic object 860. The structured data may include, for example, facts and numerical information as related to known medications, lab results, and other structured information that may be directly inserted into a form, added as a universal resource locator (URL) link, etc.

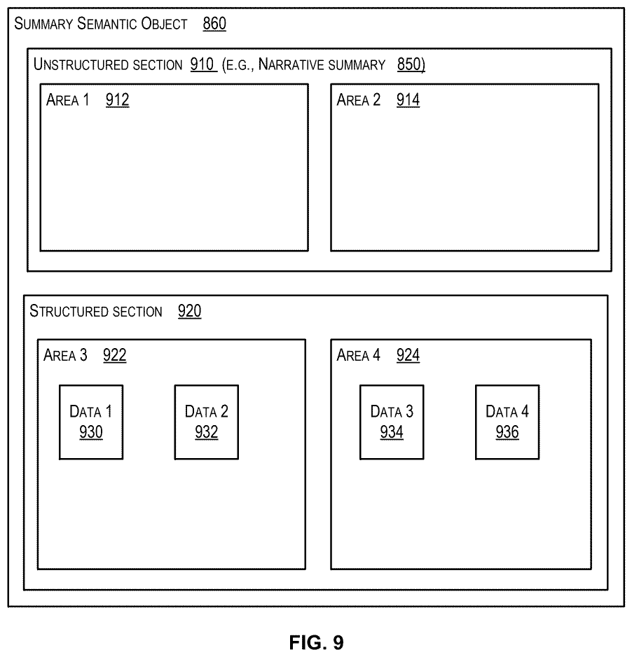

An example presentation format of summary semantic object 860 is shown in FIG. 9. As shown in FIG. 9, summary semantic object 860 includes an unstructured section 910 (including, as an example, narrative summary 850 of FIG. 8). Unstructured section 910 may include one or more areas (shown as area 1 (912) and area 2 (914)), into which narrative related to specific topics may be inserted. For instance, area 1 (912) may be used to present a general summary of a patient's current chief complaints, while area 2 (914) may be reserved for a summary of patient's family, conditions, and medication history. The information presented in unstructured section 910 may be an extract from narrative summary 850 or independently populated using extracted IR 810. Unstructured section 910 may include a generated summary of the relevant information in natural language format, bullet points of key information, extracts from notes from previous patient encounters, electronic scans of historical notes, and other long-form information.

Summary semantic object 860 may additionally include a structured section 920. In the illustrative example, structured section 920 may include one or more areas (shown as area 3 (922) and area 4 (924)), in which structured data such as lab test results and medication lists may be presented. Area 3 (922) and area 4 (924) may include structured data extracted from IR 210 used to populate an predetermined template. For example, area 3 (922) may include data 1 (930) presented as a list of past patient conditions next to data 2 (932), including a list of medications relevant to the past patient conditions, or a list of previous Assessment & Plans (A&Ps) as frequently referenced in patient charts. Similarly, area 4 (924) may include data 3 (934), with a graph visually showing the evaluated numbers from a series of blood test results, along with data 4 (936) with a list of links containing URLs linking to external or internal repository of information related to explanation of the blood test results. Such visual representation of structured data may assist the healthcare provider in identifying trends as related to the rest of the patient medical history. Optionally, the information presented in the structured section may have been further processed, for example, to emphasize the most recently entered information, such as the list of active prescriptions or lab test results over the past six months. In this way, summary semantic object 860 presents a snapshot of the patient's past history in a compact format, suitable for display on a small screen such as a tablet, as well as links to additional information of so desired. In some implementations, one or more portions of summary semantic object 860 may be reserved to allow the healthcare provider to enter additional notes.

In certain embodiments, summary semantic object 860 may include a search field or a user interface “button” to allow the healthcare provider to regenerate the summary semantic object based on any newly added information and the previously extracted IR 810. If necessary, additional information may be pulled from one or more databases to be added to IR 810. In certain embodiments, the healthcare provider may be presented with an option to save the current summary report to a file folder in memory, prior to generating an updated summary report. For instance, as a particular patient may present with multiple medical conditions, additional and/or updated summary reports may be necessary for a complete assessment of the patient history. In embodiments, one of the areas of summary semantic object 860 may be reserved for presenting recommendations for next steps of action for the patient. Such recommendations may be generated, for example, based on prior knowledge of the patient history, industry standard courses of action, latest guidance from regulatory and industry standard organizations, and others. As additional examples, certain portions of the unstructured section may be enhanced with structured information, such as URL links to access external documents (e.g., images of handwritten notes).

### Semantic Knowledge Graph

As discussed above, the clinical summary generation may process patient-level and encounter-level data through a transform layer and an enrichment layer. In some implementations, as discussed above, the transform layer performs an initial filtering of information relevant to the summarization task, such as identifying medication-related data and condition-related data and, where appropriate, extracting subsets thereof based on configured criteria. The enrichment layer can then apply knowledge graph-based techniques and other techniques to evaluate relationships among the identified clinical entities (e.g., between a patient's medications and conditions) to determine which items are contextually relevant to the summary, and to prioritize or de-emphasize items accordingly.

The knowledge graph-based techniques may rely on one or more proprietary and/or publicly accessible knowledge sources, such as medical ontologies. An ontology, as used herein, generally refers to a formal representation of domain knowledge that defines concepts, relationships, attributes, and, in some implementations, constraints, rules, or axioms, and may be utilized to construct knowledge graphs that interlink diverse information and support inference of relationships that are not explicitly stated in the source data. Coverage gaps can occur in ontologies, including missing concepts, incomplete or unidirectional relationships, insufficient granularity for subtypes, or uneven domain coverage. Such gaps can result in knowledge graphs that omit relationship information needed for particular clinical summarization use cases. Consequently, when enrichment relies on knowledge graphs affected by these gaps, the system may fail to surface certain clinically relevant connections or may underrepresent related entities, thereby diminishing the effectiveness of the enrichment layer and the quality of generated clinical summaries.

The techniques described herein address these and other challenges by providing computer-implemented systems and methods that construct, augment, and serve semantic knowledge graphs with improved recall and coverage across a broad range of clinical concepts. In some implementations, a semantic knowledge graph (SKG) is generated and maintained via a three-stage pipeline that aggregates relationship information from multiple proprietary and/or publicly accessible knowledge sources and leverages semantic objects present in patient data to form a personal knowledge graph aligned to a given patient. In a first, low-fidelity, higher-latency discovery stage, one or more LLMs and complementary inference techniques may be applied to assess potential relationships between objects for which discretely documented links are absent in available sources, thereby producing candidate relationships for new or rare queries. Candidate relationships can be persisted to a second, medium-fidelity, lower-latency validation stage, where fidelity may be increased through automated evaluation using an LLM and, in some implementations, triaged for human curation by terminologists and/or informaticists. Relationships that satisfy curation criteria may then transition to a third, high-fidelity, low-latency serving stage, from which they can be consumed by the enrichment layer to inform clinical summarization. Even where human-curated knowledge exists, the SKG may supplement relationship types and other metadata through LLM-assisted extraction from reference texts or other sources. Each persisted relationship may include metadata such as source attribution identifying one or more human-curated sources and/or LLMs, as well as applicable relationship types, and users can specify which sources and relationship types are to be applied for a given task. Different sources may contribute different enrichments that feed back to SKG consumers and a Semantic Index. To accommodate evolving clinical knowledge and long-tail queries, the system can compute new relationships opportunistically or on demand and incorporate them into the appropriate pipeline stage, thereby providing low-latency access to known, curated, and previously computed relationships while enabling ongoing augmentation.

As used herein, a “semantic knowledge graph” or SKG refers to a machine-interpretable data structure that represents healthcare, medical, and clinical entities, and equivalents thereof, as nodes and relationships among such entities as edges, where the nodes and/or edges are associated with explicit semantics that provide standardized meaning independent of any particular application. Entities may include, without limitation, patients, caregivers, providers, provider organizations, encounters, episodes of care, diagnoses, conditions, symptoms, procedures, observations, laboratory tests and results, imaging studies, medications and administrations, devices and implants, care plans, guidelines, outcomes, adverse events, social determinants of health, claims, authorizations, consents, clinical trials, cohorts, and registries. The semantics may include, without limitation, typed relationships, controlled vocabularies, clinical terminologies, taxonomies, schemas, rules, constraints, or axioms that define or constrain how entities and relationships are identified, classified, linked, inferred, validated, or harmonized across systems. The SKG may support reasoning or inference over asserted and/or derived clinical facts; may incorporate temporal, contextual, and quantitative qualifiers (for example, onset time, severity, dosage, laterality, confidence values); may include metadata such as provenance, lineage, audit trails, consent status, access-control attributes, de-identification state, timestamps, or jurisdictional constraints; and may be persisted in or implemented using any suitable store or representation, including but not limited to graph databases, relational databases, key-value stores, document stores, distributed object stores, or data lake architectures, and any data model or serialization, including but not limited to RDF-based models, property-graph models, JSON-LD, structures compatible with HL7 FHIR, or proprietary formats. The graph may be centralized or federated; materialized or virtual; static or dynamically updated; and may integrate data from electronic health records, laboratory information systems, imaging archives, pharmacy systems, claims platforms, wearable and remote monitoring devices, research systems, public health feeds, or other sources without departing from this definition. In some implementations, the SKG can be generated and continuously maintained by the SKG service 1006 using one or more knowledge sources included in the knowledge sources 1014 and one or more LLMs included in the LLMs 1016, thereby enabling dynamic incorporation of authoritative ontologies and literature together with LLM-assisted discovery and refinement while preserving explicit semantics and metadata throughout.

In some implementations, the workflow for the flow 1000 includes, but is not limited to: extracting codified clinical entities from structured and unstructured data sources using an entity linking service; mapping the extracted entities to standard coding systems; identifying relationships between the mapped entities using a combination of human-curated knowledge sources and LLMs; persisting the identified relationships in a graph database; augmenting the relationships with metadata including source attribution; and continuously updating the graph with newly discovered relationships through lazy computation based on ongoing data input and evaluation.

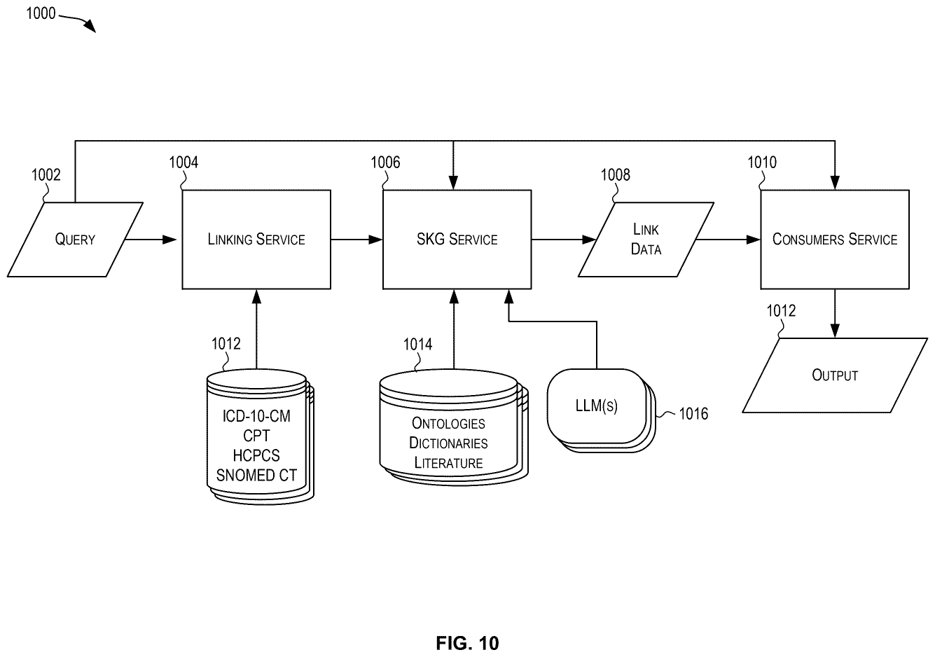

FIG. 10 is a simplified block diagram illustrating an example of a flow 1000 for generating and utilizing a SKG. The flow 1000 is executed by a service of the cloud service provider platform 514 such as the digital assistant service 518. As shown in FIG. 10, the flow 1000 begins with a query 1002. In some implementations, the query 1002 is received from a client device such as a client device of the client devices 510. In some implementations, the query concerns a patient and includes a request to generate clinical summary for the patient and/or a request for information from a chart of the patient or EHR record of the patient. In some implementations, the query can be associated with a patient's visit to a healthcare provider and describe the patient's reasons for their visit (RFV) to the healthcare provider and chief complaint (CC).

The flow 1000 continues with the linking service 1004 which can process the query 1002 to identify entities (e.g., medication entities and conditions entities) in the query 1002 and codify the identified entities using one or more coding systems included in the coding systems 1012. Examples of coding systems that can be used include, but are not limited to: ICF-10-CM, CPT; HCPCS; and SNOMED CT. In some implementations, the linking service 1004 processes the query 1002 to identify strings within the query 1002 corresponding to the patient's RFV and CC and codes the identified strings using one or more coding systems included in the coding systems 1012. In some implementations, a subset of coding systems included in the coding systems 1012 can be used to code the RFV and CC strings (e.g., ICD-10-CM, SNOMED CT), another subset of coding systems included in the coding systems 1012 can be used to code the text in the query 1002 corresponding to the patient's conditions (e.g., ICD-10-CM), and another subset of coding systems included in the coding systems 1012 can be used to code the text in the query 1002 corresponding to the patient's medications (e.g., RXNORM). In some implementations, the subset can include one or more coding systems, and coding systems within the subsets can be the same as each other and/or different from each other. The linking service 1004 can generate a data structure that includes the identified entities and the codes extracted from the coding systems 1012 for those identified entities. In some implementations, the data structure can be compliant with a standard for exchanging healthcare information electronically (e.g., a Fast Healthcare Interoperability Resources or FHIR-compliant data structure).

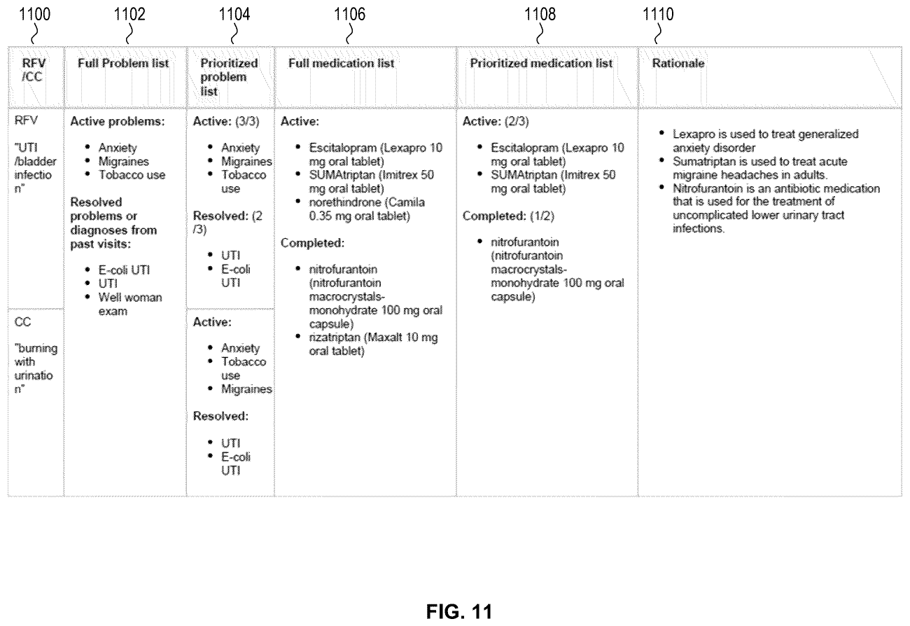

The flow 1000 continues with the SKG service 1006 which can process the query 1002 and the data structure using one or more knowledge sources included in knowledge sources 1014 and one or more LLMs included in LLMs 1016 to generate a semantic knowledge graph (SKG) and generate link data 1008 for the query 1002 using the SKG. In some implementations, the SKG can be used to prioritize and filter the coded entities provided by the linking service 1004 in which the link data 1008 can represent the prioritized and filtered coded entities. For example, as shown in FIG. 11, which illustrates an example of entities that have been prioritized and filtered using the SKG, for a given query describing the entities 1100 “UTI/bladder infection” as the RFV and “burning with urination” as the CC, the SKG service 1006 can employ the SKG to identify a full problem list 1102 for the RFV/CC, a prioritized problem list 1104 which includes filtered and prioritized problems from the full problem list 1102, a full medication list 1106 for the RFV/CC, a prioritized medication list 1108 which includes filtered and prioritized medications from the full medication list 1106, and a rationale 1108 for the prioritization and the filtering.

To generate the SKG, a three-stage approach can be employed. The first stage can be a low-fidelity, high-latency stage capable of assessing possible relationships between two objects for which there are no existing, discretely documented relationships in available knowledge sources. This stage can serve as a primary entry point for new relationships that the SKG can discover, using an LLM to respond to new and rare queries. These newly discovered relationships can be persisted in a second stage in which a medium-fidelity, low-latency stage is maintained for future use. The fidelity of relationships in this stage can be improved through automatic evaluation using an LLM. Relationships from the second stage may be prioritized for human curation by terminologists and/or informaticists, and once they have been reviewed can enter the final, high-fidelity, low-latency stage. It is important to note that, even for existing human-curated knowledge, gaps in relationship types and other metadata can necessitate the SKG supplementing with information that the LLM either already encodes or may help to extract from reference texts. Thus, the SKG can serve known, curated, and previously computed relationships with low latency. Every relationship can contain metadata including source attribution (e.g., human curated source, which LLM, etc.), including the possibility of identifying multiple sources. Users can specify which sources and relationship types are of interest. Persisted relationships can include all source attributes, and different sources can provide different enrichments that can ultimately feed back to consumers of the SKG. The sources of relationship information for the SKG can include the knowledge sources 1014 and outputs produced by the LLMs 1016. LLMs can enhance the SKG by suggesting additional relationships that can be further curated. In some implementations, a lazy-compute approach can be implemented to continuously add new relationships, with the SKG service 1006 orchestrating LLM-assisted discovery, validation, and promotion of relationships across stages while maintaining provenance and confidence metadata.

In some implementations, the SKG can be generated using data retrieved from the knowledge sources 1014 and/or outputs produced by the LLMs 1016. One or more ontologies included in the knowledge sources 1014 can provide the machine-interpretable specification of the domain vocabulary and relationships (e.g., classes, properties, constraints, and mappings to clinical terminologies such as SNOMED CT, LOINC, or RxNorm). Source data can be transformed and normalized to ontology-aligned representations using any suitable approach, including structured mappings, virtualization, or extract-transform-load pipelines. As part of this process, entities (e.g., patients, encounters, observations, medications, procedures) are identified and de-duplicated, relationships are asserted and typed, and temporal and contextual qualifiers (e.g., effective time, severity, dosage, laterality), provenance, consent and access-control attributes, and confidence scores are associated as metadata. The resulting nodes and edges may be materialized in a graph store or exposed virtually, and may be expressed in any suitable serialization or data model. Constraint and validation artifacts (e.g., shape definitions using any suitable formalism) may be applied to detect incompleteness or inconsistency, and the ontology layer may be modular or federated to support multiple specialties, institutions, or jurisdictions. The foregoing steps may be performed in any suitable order, combination, iteratively or in parallel, and not all steps are required in all implementations. In some implementations, one or more LLMs included in the LLMs 1016 can be used to assist with entity and relation extraction from unstructured text, ontology-aligned normalization and mapping, de-duplication heuristics, and assignment or calibration of confidence values, thereby improving coverage and harmonization across the knowledge sources 1014 while deferring to the authoritative semantics provided by the knowledge sources 1014 and associated ontologies.

The graph may be incrementally enriched and modified over time through ontology-driven reasoning and rule evaluation, as well as through the incorporation of additional data and outputs from analytics or generative machine learning models such as LLMs 1016. By way of non-limiting example, model outputs such as entity and relation extractions from unstructured text, phenotyping classifications, risk scores, cohort assignments, image-derived measurements, or temporal trend detections may be ingested as new facts, annotations, or inferred relationships, each optionally accompanied by provenance, versioning, and confidence metadata. Updates to the ontologies or value sets may trigger reclassification or remapping of existing nodes and edges, while change-management mechanisms support backward-compatible evolution, conflict resolution, and, where appropriate, retraction of stale or superseded assertions. This continuous, policy- and ontology-governed refinement permits the SKG to adapt to newly available data, evolving clinical knowledge, and improved models, without limiting the system to any particular storage technology, inference engine, mapping formalism, or learning approach. In some implementations, the SKG service 1006 can leverage one or more LLMs included in the LLMs 1016 to dynamically and continuously update relationships, enrich metadata, and prompt re-evaluation when new knowledge sources 1014 or model outputs become available, thereby sustaining low-latency access to curated and computed relationships while maintaining traceability through comprehensive provenance and versioning attributes.

In some implementations, the link data 1008 can be a data structure that includes the prioritized and filtered coded entities generated by the SKG service 1006. The data structure can describe the following relationships: RFV/CC-Condition; RFV/CC-Medication; and Conditions-Medications. RFV/CC Condition represents the contextual information contained within either RFV or CC for the patient's visit and the candidate conditions from patient's medical history that have been prioritized and filtered using the SKG. RFV/CC Medications represents the contextual information contained within either RFV or CC for the patient's visit and the candidate medications from patient's medical history that have been prioritized and filtered using the SKG. Conditions-Medications represents the links between list of conditions from patient's medical history and the list of candidate medications according to the desired set of relationship types (e.g., “may treat,” “may cause,” etc.) using the SKG.

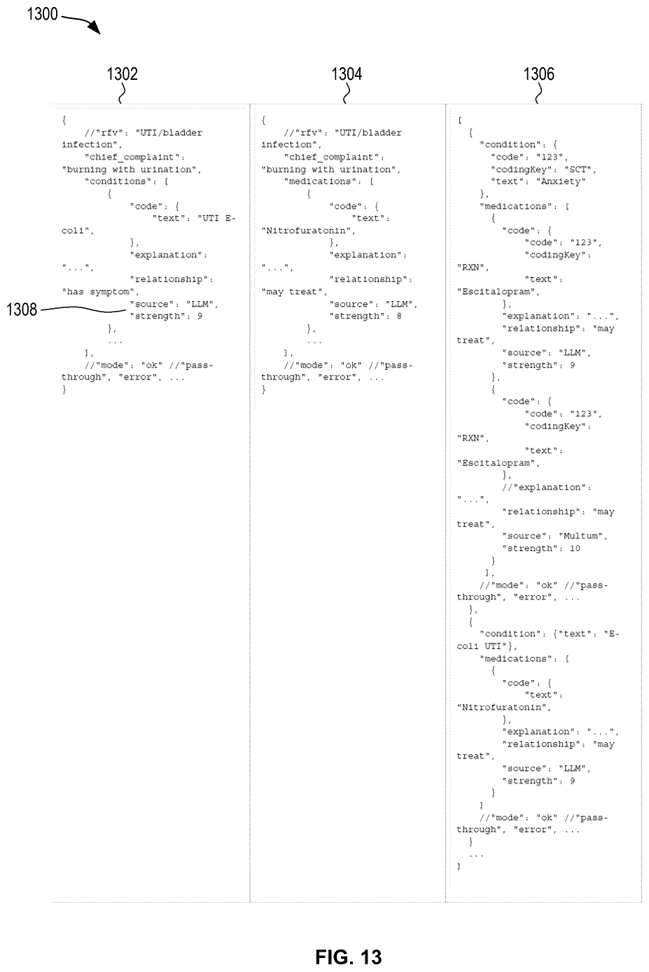

FIG. 12 illustrates an example of data structure 1200 generated using a SKG. As shown in FIG. 12, for the given query describing the entities “UTI/bladder infection” as the RFV and “burning with urination” as the CC, the data structure 1200 includes RFV/CC Condition 1202 for prioritized and filtered coded entities pertaining to conditions associated with the RFV/CC, RFV/CC Medication 1204 for prioritized and filtered coded entities pertaining to medications associated with the RFV/CC, and Conditions-Medications 1206 for relationships between the RFV/CC Condition 1202 and RFV/CC Medication 1204. As described above, the SKG service 1006 can leverage one or LLMs to enhance the SKG. FIG. 13 illustrates another example of a data structure 1300 generated using an enhanced SKG. As shown in FIG. 13, for the given query describing the entities “UTI/bladder infection” as the RFV and “burning with urination” as the CC, the data structure 1300 includes RFV/CC Condition 1302 for prioritized and filtered coded entities pertaining to conditions associated with the RFV/CC, RFV/CC Medication 1304 for prioritized and filtered coded entities pertaining to medications associated with the RFV/CC, and Conditions-Medications 1306 for relationships between the RFV/CC Condition 1302 and RFV/CC Medication 1304. As further shown in FIG. 13, the data structure 1300 can identify a source 1308 of the relationship (e.g., LLM).

The flow 1000 continues with the Consumers Service 1010 which can process the link data 1008 and generate an output 1012. As described above, with respect to FIGS. 8 and 9, the output 1012 can be a clinical summary. Another example of an output can be a response for a conversational chart search that provides an answer to the query by performing a search over the patient's EHR. The link data 1008 can assist conversational chart search by providing a comprehensive understanding of a healthcare domain and conceptual data model underlying the patient's EHR.

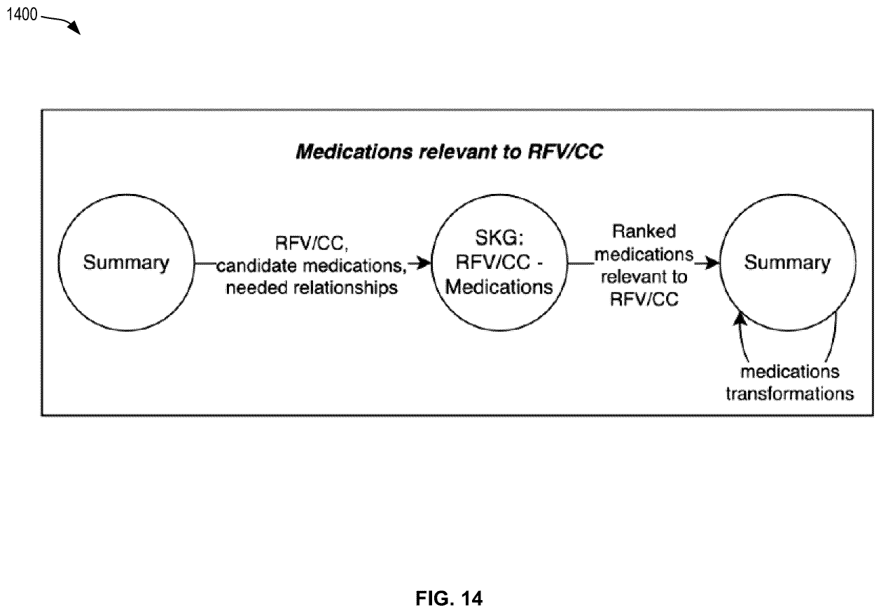

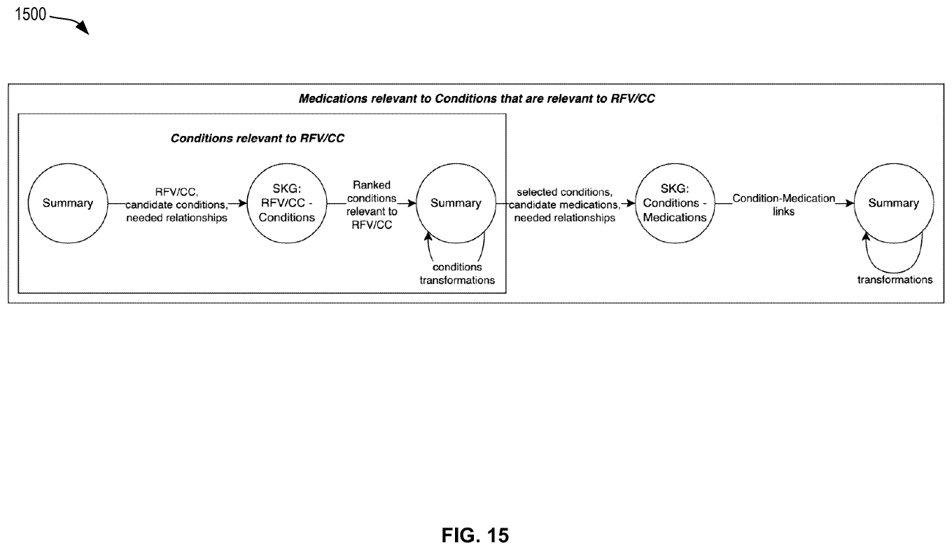

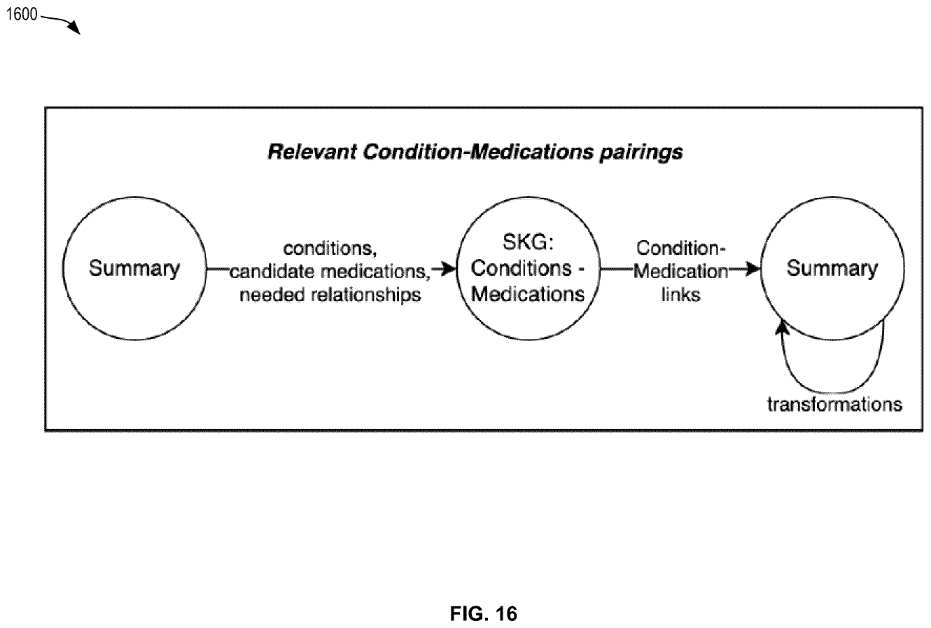

Examples of pathways for utilizing the SKG of the SKG service 1006 include: a pathway in which the patient's conditions that are relevant to the patient's RFV or CC (e.g., RFV/CC Condition) are obtained; pathway 1400 of FIG. 14 in which the patient's medications that are relevant to the patient's RFV or CC (e.g., direct RFV/CC Medication) are obtained; pathway 1500 of FIG. 15 in which the patient's medications that are relevant to the conditions that are relevant to the RFV or CC (e.g., indirect RFV/CC Medication) are obtained; and pathway 1600 of FIG. 16 in which the patient's conditions and medications (e.g., Conditions-Medications) are linked.

For the RFV/CC Condition pathway, the patient's conditions can be obtained using links from knowledge sources and LLM-generated links. In the case of the LLM-generated links, the strings in the query corresponding to the patient's RFV/CC and conditions are input to the LLM along with the potential types of condition-condition relationships (e.g., “is subtype of”, “is supertype of”, “symptom of”, “has symptom”, “can cause”, “can be caused by”, “risk factor for”, “has risk factor of”, “precedes”, “follows”, “complicates treatment”, “mimics”, “excludes”, “is synonym of”, “frequently co-occurs with”).

For the direct RFV/CC Medication pathway, the patient's medications that are relevant to the patient's RFV or CC can be obtained using LLM-generated links. In the case of the LLM-generated links, the strings in the query corresponding to the patient's RFV/CC and medications are input to the LLM along with the potential types of medication-condition relationships (e.g., “may treat”, “may cure”, “adjunctive therapy”, “maintenance”, “symptom management”, “may prevent”, “may prevent complications”, “supplemental support”, “may diagnose”, “off-label treatment”, “may cause”, “contraindicated”).

For the Conditions-Medications pathway, the link between the patient's conditions and medications can be obtained from knowledge sources and using LLM-generated links. In the case of the LLM-generated links, the conditions and medications identified in the other pathways are used as an input to the LLM along with the potential types of links (e.g., “may treat” and “contraindicated”). The conditions can be passed to the LLM as a single list or one-at-a-time

For the indirect RFV/CC pathway, the RFV/Condition pathway can be utilized followed by the Conditions-Medications pathway.

### Example Scenario

A patient has an upcoming visit with an acute reason for which a pre-visit clinical summary is desired. Prior to the patient's arrival, the patient's RFV would be available (e.g., from appointment scheduling). To generate the pre-visit summary, the flow 1000 would use the patient's RFV to contextualize the prioritization and filtering of conditions and medications with help of the SKG service 1006. The patient's CC becomes available after the patient has been seen by a healthcare provider. In this case, to generate the clinical summary, the flow 1000 would use the patient's RFV and CC to contextualize the prioritization and filtering of conditions and medications and identify relationships between conditions and medications with help of the SKG service 1006.

### Illustrative Methods

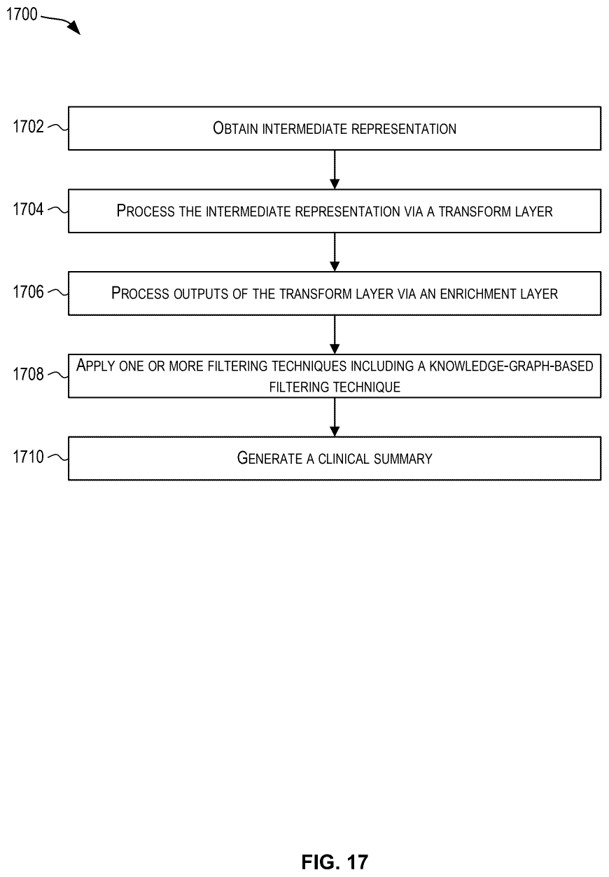

FIG. 17 depicts an example of a process 1700 for generating a clinical summary using an SKG. The process depicted in FIG. 17 may be implemented in software (e.g., code, instructions, program) executed by one or more processing units (e.g., processors, cores) of the respective systems, hardware, or combinations thereof. The software may be stored on one or more non-transitory storage media (e.g., on a memory device). The process shown in FIG. 17 and described below is intended to be illustrative and non-limiting. Although FIG. 17 depicts the various steps occurring in a particular sequence or order, this is not intended to be limiting. In certain alternative embodiments, the steps may be performed in some different order, or some steps may be performed in parallel. In certain embodiments, such as in the embodiment depicted in FIGS. 5-7, the process shown FIG. 17 may be performed by the digital assistant service 518.

At block 1702, an intermediate representation of patient-specific data is obtained. In some implementations, the intermediate representation is obtained in response to a clinical query associated with an anticipated or ongoing patient encounter. The intermediate representation can include semantic objects extracted from one or more data sources. The semantic objects can include a condition and a medication associated with a patient, and the intermediate representation can further represent a reason for visit and a chief complaint for the encounter. In some implementations, the intermediate representation is obtained by receiving the clinical query, identifying, in the clinical query, clinical entities including at least medication entities and condition entities, and codifying the clinical entities using one or more coding systems to form a data structure compliant with an electronic healthcare information exchange standard.

At block 1704, the intermediate representation is processed. In some implementations, the intermediate representation is processed via a transform layer. The transform layer can include multiple filtering modules to extract condition-related and medication-related information relevant to the encounter.

At block 1706, outputs of the transform layer are processed via an enrichment layer. In some implementations, processing the outputs further filters and contextualize subsets of the condition-related and medication-related information. In some implementations, processing the outputs includes extracting a subset of medications known to have therapeutic or adverse effects with respect to filtered conditions and extracting a subset of conditions affiliated with the subset of medications.

At block 1708, one or more filtering techniques are applied. In some implementations, the one or more filtering techniques are applied within the enrichment layer. In some implementations, the one or more filtering techniques include a knowledge-graph-based filtering technique that prioritizes and retains entities according to a relevancy score. In some implementations, applying the knowledge-graph-based filtering technique includes utilizing a semantic knowledge graph to prioritize and filter coded entities identified from the query and the intermediate representation to produce link data comprising at least a prioritized condition list, a prioritized medication list, and relationships among conditions and medications. In some implementations, the link data includes relationship sets that include: relationships that link a reason for visit or a chief complaint to candidate conditions relevant to encounter context; relationships that link the reason for visit or the chief complaint directly to candidate medications relevant to context of the encounter; relationships that link candidate conditions to candidate medications using relationship types; and relationships obtained indirectly by first identifying candidate conditions and then linking the candidate conditions to candidate medications.

At block 1710, a clinical summary for the patient is generated. In some implementations, the clinical summary is generated based on outputs of the transform layer and the enrichment layer. In some implementations, the clinical summary includes a narrative summary generated using natural language processing and a structured summary comprising structured facts derived from the intermediate representation. In some implementations, generating the clinical summary includes: populating a summary data structure with the link data such that a narrative portion of the clinical summary describes, in natural language, a context of the encounter and clinically prioritized entities and a structured portion of the clinical summary presents structured facts selected from the prioritized condition list, the prioritized medication list, and links that explain a rationale for prioritization and filtering. In some implementations, the link data can be used to guide selection of clinically relevant entities for inclusion in the clinical summary.

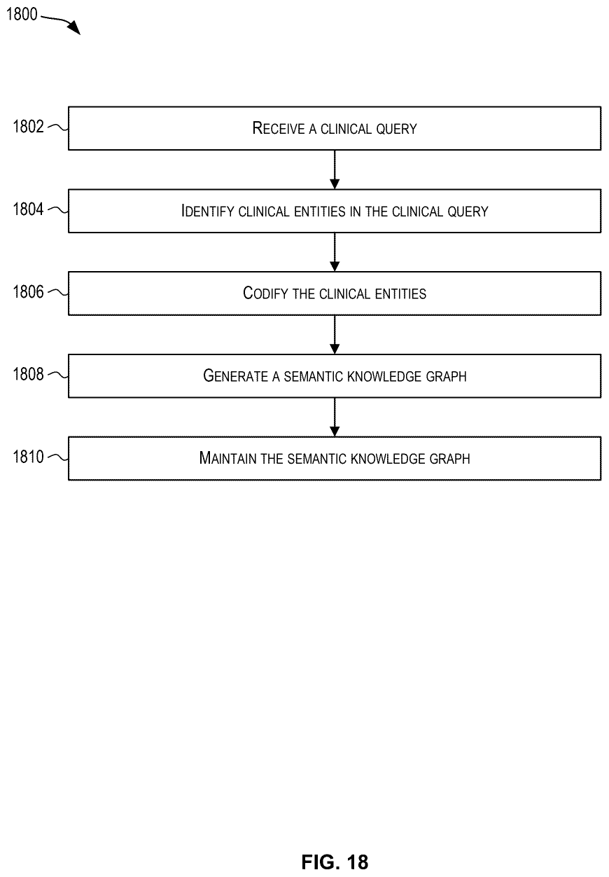

FIG. 18 depicts an example of a process 1800 for generating and maintaining an SKG. The process depicted in FIG. 18 may be implemented in software (e.g., code, instructions, program) executed by one or more processing units (e.g., processors, cores) of the respective systems, hardware, or combinations thereof. The software may be stored on one or more non-transitory storage media (e.g., on a memory device). The process shown in FIG. 18 and described below is intended to be illustrative and non-limiting. Although FIG. 18 depicts the various steps occurring in a particular sequence or order, this is not intended to be limiting. In certain alternative embodiments, the steps may be performed in some different order, or some steps may be performed in parallel. In certain embodiments, such as in the embodiment depicted in FIGS. 5-7, the process shown FIG. 18 may be performed by the digital assistant service 518.

At block 1802, a clinical query is received. In some implementations, the query is received from a client device such as a client device of the client devices 510. In some implementations, the query concerns a patient and includes a request to generate clinical summary for the patient and/or a request for information from a chart of the patient or EHR record of the patient. In some implementations, the query can be associated with a patient's visit to a healthcare provider and describe the patient's reasons for their visit (RFV) to the healthcare provider and chief complaint (CC).

At block 1804, clinical entities in the clinical query are identified. In some implementations, the clinical entities include at least medication entities and condition entities.

At block 1806, the clinical entities are codified. In some implementations, the clinical entities are codified using one or more coding systems. In some implementations, the codified clinical entities are included in a data structure compliant with an electronic healthcare information exchange standard.

At block 1808, a semantic knowledge graph is generated. In some implementations, the semantic knowledge graph is generated using one or more knowledge sources and LLMs. In some implementations, a semantic knowledge graph represents clinical entities and typed relationships among the clinical entities. In some implementations, generating the semantic knowledge graph includes: executing a multi-stage pipeline including a discovery stage that applies a large language model to propose candidate relationships between clinical entities for which discretely documented links are absent in available knowledge sources; validating the candidate relationships; and persisting, for each candidate relationship of the candidate relationships, metadata comprising at least a source attribution that identifies a large language model, and a relationship type.

At block 1812, the semantic knowledge graph is maintained. In some implementations, maintaining the semantic knowledge graph includes: normalizing source data to ontology-aligned representations; applying constraint and validation artifacts to detect incompleteness or inconsistency and reclassifying nodes or edges when ontologies or value sets are updated; and incrementally enriching the semantic knowledge graph over time by ingesting outputs of a large language model.

### Examples Of Cloud Infrastructure Architectures

The term cloud service is generally used to refer to a service that is made available by a cloud service provider (CSP) to users (e.g., cloud service customers) on demand (e.g., via a subscription model) using systems and infrastructure (cloud infrastructure) provided by the CSP. Typically, the servers and systems that make up the CSP's infrastructure are separate from the user's own on-premise servers and systems. Users can thus avail themselves of cloud services provided by the CSP without having to purchase separate hardware and software resources for the services. Cloud services are designed to provide a subscribing user easy, scalable access to applications and computing resources without the user having to invest in procuring the infrastructure that is used for providing the services.

There are several cloud service providers that offer various types of cloud services. As discussed herein, there are various types or models of cloud services including IaaS, software as a service (SaaS), platform as a service (PaaS), and others. A user can subscribe to one or more cloud services provided by a CSP. The user can be any entity such as an individual, an organization, an enterprise, and the like. When a user subscribes to or registers for a service provided by a CSP, a tenancy or an account is created for that user. The user can then, via this account, access the subscribed-to one or more cloud resources associated with the account.

As noted above, IaaS is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

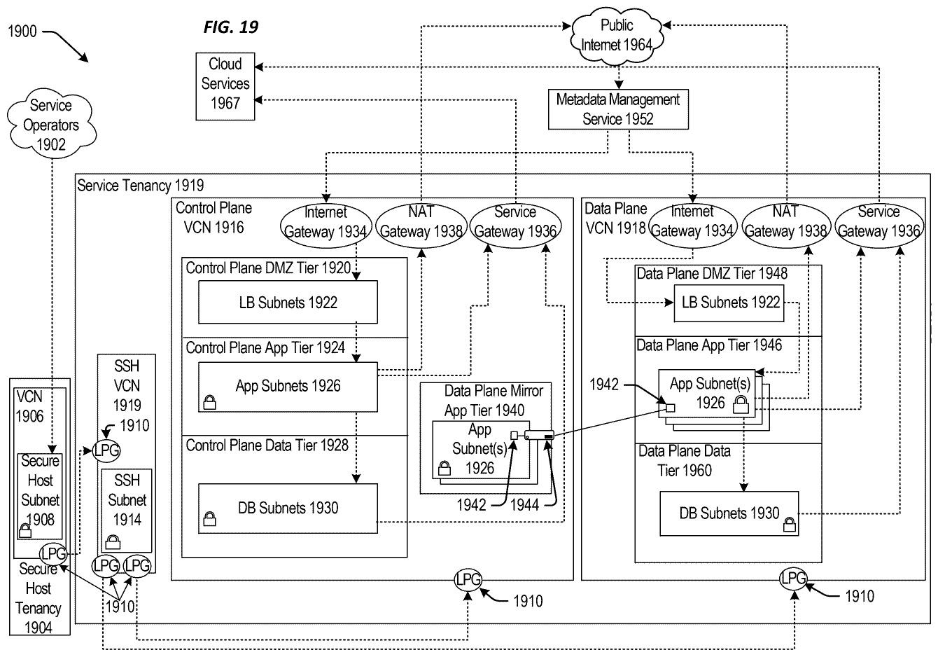

FIG. 19 is a block diagram 1900 illustrating an example pattern of an IaaS architecture, according to at least one embodiment. Service operators 1902 can be communicatively coupled to a secure host tenancy 1904 that can include a virtual cloud network (VCN) 1906 and a secure host subnet 1908. In some examples, the service operators 1902 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1906 and/or the Internet.

The VCN 1906 can include a local peering gateway (LPG) 1910 that can be communicatively coupled to a secure shell (SSH) VCN 1912 via an LPG 1910 contained in the SSH VCN 1912. The SSH VCN 1912 can include an SSH subnet 1914, and the SSH VCN 1912 can be communicatively coupled to a control plane VCN 1916 via the LPG 1910 contained in the control plane VCN 1916. Also, the SSH VCN 1912 can be communicatively coupled to a data plane VCN 1918 via an LPG 1910. The control plane VCN 1916 and the data plane VCN 1918 can be contained in a service tenancy 1919 that can be owned and/or operated by the IaaS provider.

The control plane VCN 1916 can include a control plane demilitarized zone (DMZ) tier 1920 that acts as a perimeter network (e.g., portions of a corporate network between the corporate intranet and external networks). The DMZ-based servers may have restricted responsibilities and help keep breaches contained. Additionally, the DMZ tier 1920 can include one or more load balancer (LB) subnet(s) 1922, a control plane app tier 1924 that can include app subnet(s) 1926, a control plane data tier 1928 that can include database (DB) subnet(s) 1930 (e.g., frontend DB subnet(s) and/or backend DB subnet(s)). The LB subnet(s) 1922 contained in the control plane DMZ tier 1920 can be communicatively coupled to the app subnet(s) 1926 contained in the control plane app tier 1924 and an Internet gateway 1934 that can be contained in the control plane VCN 1916, and the app subnet(s) 1926 can be communicatively coupled to the DB subnet(s) 1930 contained in the control plane data tier 1928 and a service gateway 1936 and a network address translation (NAT) gateway 1938. The control plane VCN 1916 can include the service gateway 1936 and the NAT gateway 1938.

The control plane VCN 1916 can include a data plane mirror app tier 1940 that can include app subnet(s) 1926. The app subnet(s) 1926 contained in the data plane mirror app tier 1940 can include a virtual network interface controller (VNIC) 1942 that can execute a compute instance 1944. The compute instance 1944 can communicatively couple the app subnet(s) 1926 of the data plane mirror app tier 1940 to app subnet(s) 1926 that can be contained in a data plane app tier 1946.

The data plane VCN 1918 can include the data plane app tier 1946, a data plane DMZ tier 1948, and a data plane data tier 1950. The data plane DMZ tier 1948 can include LB subnet(s) 1922 that can be communicatively coupled to the app subnet(s) 1926 of the data plane app tier 1946 and the Internet gateway 1934 of the data plane VCN 1918. The app subnet(s) 1926 can be communicatively coupled to the service gateway 1936 of the data plane VCN 1918 and the NAT gateway 1938 of the data plane VCN 1918. The data plane data tier 1950 can also include the DB subnet(s) 1930 that can be communicatively coupled to the app subnet(s) 1926 of the data plane app tier 1946.

The Internet gateway 1934 of the control plane VCN 1916 and of the data plane VCN 1918 can be communicatively coupled to a metadata management service 1952 that can be communicatively coupled to public Internet 1954. Public Internet 1954 can be communicatively coupled to the NAT gateway 1938 of the control plane VCN 1916 and of the data plane VCN 1918. The service gateway 1936 of the control plane VCN 1916 and of the data plane VCN 1918 can be communicatively coupled to cloud services 1956.

In some examples, the service gateway 1936 of the control plane VCN 1916 or of the data plane VCN 1918 can make application programming interface (API) calls to cloud services 1956 without going through public Internet 1954. The API calls to cloud services 1956 from the service gateway 1936 can be one-way: the service gateway 1936 can make API calls to cloud services 1956, and cloud services 1956 can send requested data to the service gateway 1936. But, cloud services 1956 may not initiate API calls to the service gateway 1936.

In some examples, the secure host tenancy 1904 can be directly connected to the service tenancy 1919, which may be otherwise isolated. The secure host subnet 1908 can communicate with the SSH subnet 1914 through an LPG 1910 that may enable two-way communication over an otherwise isolated system. Connecting the secure host subnet 1908 to the SSH subnet 1914 may give the secure host subnet 1908 access to other entities within the service tenancy 1919.

The control plane VCN 1916 may allow users of the service tenancy 1919 to set up or otherwise provision desired resources. Desired resources provisioned in the control plane VCN 1916 may be deployed or otherwise used in the data plane VCN 1918. In some examples, the control plane VCN 1916 can be isolated from the data plane VCN 1918, and the data plane mirror app tier 1940 of the control plane VCN 1916 can communicate with the data plane app tier 1946 of the data plane VCN 1918 via VNICs 1942 that can be contained in the data plane mirror app tier 1940 and the data plane app tier 1946.

In some examples, users of the system, or customers, can make requests, for example create, read, update, or delete (CRUD) operations, through public Internet 1954 that can communicate the requests to the metadata management service 1952. The metadata management service 1952 can communicate the request to the control plane VCN 1916 through the Internet gateway 1934. The request can be received by the LB subnet(s) 1922 contained in the control plane DMZ tier 1920. The LB subnet(s) 1922 may determine that the request is valid, and in response to this determination, the LB subnet(s) 1922 can transmit the request to app subnet(s) 1926 contained in the control plane app tier 1924. If the request is validated and requires a call to public Internet 1954, the call to public Internet 1954 may be transmitted to the NAT gateway 1938 that can make the call to public Internet 1954. Metadata that may be desired to be stored by the request can be stored in the DB subnet(s) 1930.

In some examples, the data plane mirror app tier 1940 can facilitate direct communication between the control plane VCN 1916 and the data plane VCN 1918. For example, changes, updates, or other suitable modifications to configuration may be desired to be applied to the resources contained in the data plane VCN 1918. Via a VNIC 1942, the control plane VCN 1916 can directly communicate with, and can thereby execute the changes, updates, or other suitable modifications to configuration to, resources contained in the data plane VCN 1918.

In some embodiments, the control plane VCN 1916 and the data plane VCN 1918 can be contained in the service tenancy 1919. In this case, the user, or the customer, of the system may not own or operate either the control plane VCN 1916 or the data plane VCN 1918. Instead, the IaaS provider may own or operate the control plane VCN 1916 and the data plane VCN 1918, both of which may be contained in the service tenancy 1919. This embodiment can enable isolation of networks that may prevent users or customers from interacting with other users′, or other customers′, resources. Also, this embodiment may allow users or customers of the system to store databases privately without needing to rely on public Internet 1954, which may not have a desired level of threat prevention, for storage.

In other embodiments, the LB subnet(s) 1922 contained in the control plane VCN 1916 can be configured to receive a signal from the service gateway 1936. In this embodiment, the control plane VCN 1916 and the data plane VCN 1918 may be configured to be called by a customer of the IaaS provider without calling public Internet 1954. Customers of the IaaS provider may desire this embodiment since database(s) that the customers use may be controlled by the IaaS provider and may be stored on the service tenancy 1919, which may be isolated from public Internet 1954.

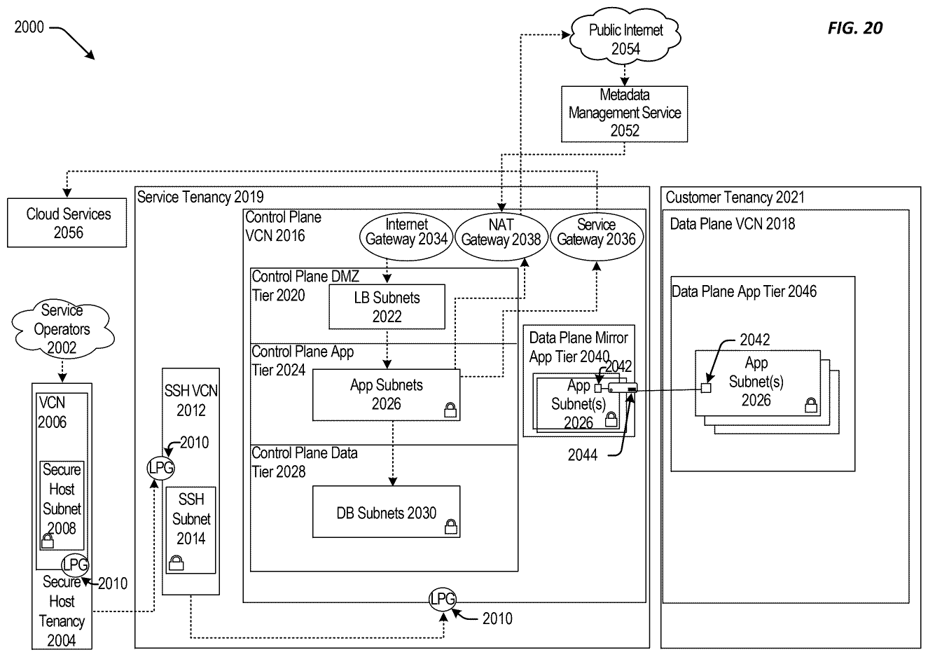

FIG. 20 is a block diagram 2000 illustrating another example pattern of an IaaS architecture, according to at least one embodiment. Service operators 2002 (e.g., service operators 1902 of FIG. 19) can be communicatively coupled to a secure host tenancy 2004 (e.g., the secure host tenancy 1904 of FIG. 19) that can include a virtual cloud network (VCN) 2006 (e.g., the VCN 1906 of FIG. 19) and a secure host subnet 2008 (e.g., the secure host subnet 1908 of FIG. 19). The VCN 2006 can include a local peering gateway (LPG) 2010 (e.g., the LPG 1910 of FIG. 19) that can be communicatively coupled to a secure shell (SSH) VCN 2012 (e.g., the SSH VCN 1912 of FIG. 19) via an LPG 1910 contained in the SSH VCN 2012. The SSH VCN 2012 can include an SSH subnet 2014 (e.g., the SSH subnet 1914 of FIG. 19), and the SSH VCN 2012 can be communicatively coupled to a control plane VCN 2016 (e.g., the control plane VCN 1916 of FIG. 19) via an LPG 2010 contained in the control plane VCN 2016. The control plane VCN 2016 can be contained in a service tenancy 2019 (e.g., the service tenancy 1919 of FIG. 19), and the data plane VCN 2018 (e.g., the data plane VCN 1918 of FIG. 19) can be contained in a customer tenancy 2021 that may be owned or operated by users, or customers, of the system.

The control plane VCN 2016 can include a control plane DMZ tier 2020 (e.g., the control plane DMZ tier 1920 of FIG. 19) that can include LB subnet(s) 2022 (e.g., LB subnet(s) 1922 of FIG. 19), a control plane app tier 2024 (e.g., the control plane app tier 1924 of FIG. 19) that can include app subnet(s) 2026 (e.g., app subnet(s) 1926 of FIG. 19), a control plane data tier 2028 (e.g., the control plane data tier 1928 of FIG. 19) that can include database (DB) subnet(s) 2030 (e.g., similar to DB subnet(s) 1930 of FIG. 19). The LB subnet(s) 2022 contained in the control plane DMZ tier 2020 can be communicatively coupled to the app subnet(s) 2026 contained in the control plane app tier 2024 and an Internet gateway 2034 (e.g., the Internet gateway 1934 of FIG. 19) that can be contained in the control plane VCN 2016, and the app subnet(s) 2026 can be communicatively coupled to the DB subnet(s) 2030 contained in the control plane data tier 2028 and a service gateway 2036 (e.g., the service gateway 1936 of FIG. 19) and a network address translation (NAT) gateway 2038 (e.g., the NAT gateway 1938 of FIG. 19). The control plane VCN 2016 can include the service gateway 2036 and the NAT gateway 2038.

The control plane VCN 2016 can include a data plane mirror app tier 2040 (e.g., the data plane mirror app tier 1940 of FIG. 19) that can include app subnet(s) 2026. The app subnet(s) 2026 contained in the data plane mirror app tier 2040 can include a virtual network interface controller (VNIC) 2042 (e.g., the VNIC of 1942) that can execute a compute instance 2044 (e.g., similar to the compute instance 1944 of FIG. 19). The compute instance 2044 can facilitate communication between the app subnet(s) 2026 of the data plane mirror app tier 2040 and the app subnet(s) 2026 that can be contained in a data plane app tier 2046 (e.g., the data plane app tier 1946 of FIG. 19) via the VNIC 2042 contained in the data plane mirror app tier 2040 and the VNIC 2042 contained in the data plane app tier 2046.

The Internet gateway 2034 contained in the control plane VCN 2016 can be communicatively coupled to a metadata management service 2052 (e.g., the metadata management service 1952 of FIG. 19) that can be communicatively coupled to public Internet 2054 (e.g., public Internet 1954 of FIG. 19). Public Internet 2054 can be communicatively coupled to the NAT gateway 2038 contained in the control plane VCN 2016. The service gateway 2036 contained in the control plane VCN 2016 can be communicatively coupled to cloud services 2056 (e.g., cloud services 1956 of FIG. 19).

In some examples, the data plane VCN 2018 can be contained in the customer tenancy 2021. In this case, the IaaS provider may provide the control plane VCN 2016 for each customer, and the IaaS provider may, for each customer, set up a unique compute instance 2044 that is contained in the service tenancy 2019. Each compute instance 2044 may allow communication between the control plane VCN 2016, contained in the service tenancy 2019, and the data plane VCN 2018 that is contained in the customer tenancy 2021. The compute instance 2044 may allow resources, that are provisioned in the control plane VCN 2016 that is contained in the service tenancy 2019, to be deployed or otherwise used in the data plane VCN 2018 that is contained in the customer tenancy 2021.

In other examples, the customer of the IaaS provider may have databases that live in the customer tenancy 2021. In this example, the control plane VCN 2016 can include the data plane mirror app tier 2040 that can include app subnet(s) 2026. The data plane mirror app tier 2040 can reside in the data plane VCN 2018, but the data plane mirror app tier 2040 may not live in the data plane VCN 2018. That is, the data plane mirror app tier 2040 may have access to the customer tenancy 2021, but the data plane mirror app tier 2040 may not exist in the data plane VCN 2018 or be owned or operated by the customer of the IaaS provider. The data plane mirror app tier 2040 may be configured to make calls to the data plane VCN 2018 but may not be configured to make calls to any entity contained in the control plane VCN 2016. The customer may desire to deploy or otherwise use resources in the data plane VCN 2018 that are provisioned in the control plane VCN 2016, and the data plane mirror app tier 2040 can facilitate the desired deployment, or other usage of resources, of the customer.

In some embodiments, the customer of the IaaS provider can apply filters to the data plane VCN 2018. In this embodiment, the customer can determine what the data plane VCN 2018 can access, and the customer may restrict access to public Internet 2054 from the data plane VCN 2018. The IaaS provider may not be able to apply filters or otherwise control access of the data plane VCN 2018 to any outside networks or databases. Applying filters and controls by the customer onto the data plane VCN 2018, contained in the customer tenancy 2021, can help isolate the data plane VCN 2018 from other customers and from public Internet 2054.

In some embodiments, cloud services 2056 can be called by the service gateway 2036 to access services that may not exist on public Internet 2054, on the control plane VCN 2016, or on the data plane VCN 2018. The connection between cloud services 2056 and the control plane VCN 2016 or the data plane VCN 2018 may not be live or continuous. Cloud services 2056 may exist on a different network owned or operated by the IaaS provider. Cloud services 2056 may be configured to receive calls from the service gateway 2036 and may be configured to not receive calls from public Internet 2054. Some cloud services 2056 may be isolated from other cloud services 2056, and the control plane VCN 2016 may be isolated from cloud services 2056 that may not be in the same region as the control plane VCN 2016. For example, the control plane VCN 2016 may be located in “Region 1,” and cloud service “Deployment 19,” may be located in Region 1 and in “Region 2.” If a call to Deployment 19 is made by the service gateway 2036 contained in the control plane VCN 2016 located in Region 1, the call may be transmitted to Deployment 19 in Region 1. In this example, the control plane VCN 2016, or Deployment 19 in Region 1, may not be communicatively coupled to, or otherwise in communication with, Deployment 19 in Region 2.

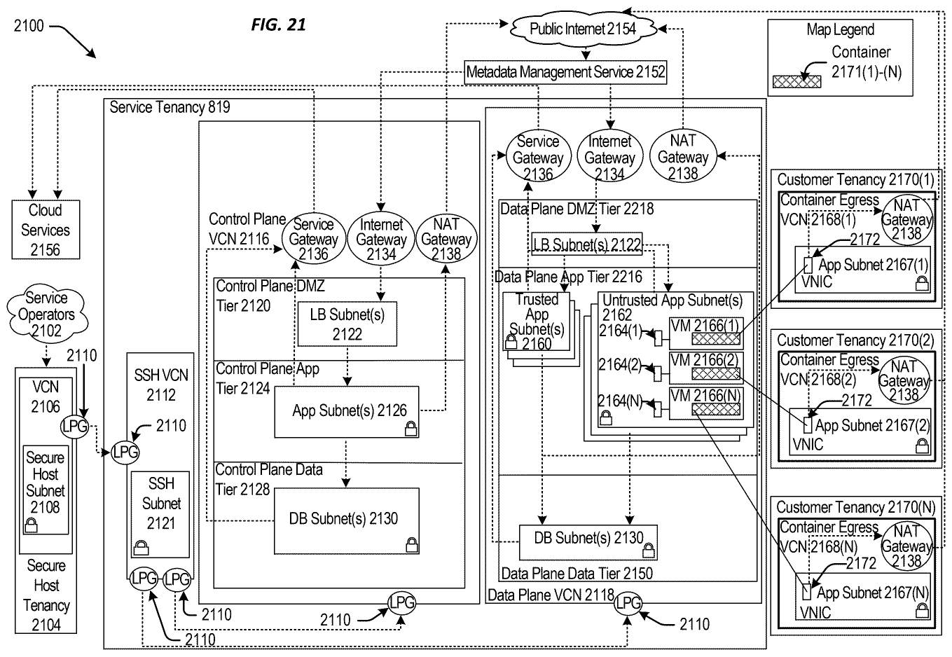

FIG. 21 is a block diagram 2100 illustrating another example pattern of an IaaS architecture, according to at least one embodiment. Service operators 2102 (e.g., service operators 1902 of FIG. 19) can be communicatively coupled to a secure host tenancy 2104 (e.g., the secure host tenancy 1904 of FIG. 19) that can include a virtual cloud network (VCN) 2106 (e.g., the VCN 1906 of FIG. 19) and a secure host subnet 2108 (e.g., the secure host subnet 1908 of FIG. 19). The VCN 2106 can include an LPG 2110 (e.g., the LPG 1910 of FIG. 19) that can be communicatively coupled to an SSH VCN 2112 (e.g., the SSH VCN 1912 of FIG. 19) via an LPG 2110 contained in the SSH VCN 2112. The SSH VCN 2112 can include an SSH subnet 2114 (e.g., the SSH subnet 1914 of FIG. 19), and the SSH VCN 2112 can be communicatively coupled to a control plane VCN 2116 (e.g., the control plane VCN 1916 of FIG. 19) via an LPG 2110 contained in the control plane VCN 2116 and to a data plane VCN 2118 (e.g., the data plane 1918 of FIG. 19) via an LPG 2110 contained in the data plane VCN 2118. The control plane VCN 2116 and the data plane VCN 2118 can be contained in a service tenancy 2119 (e.g., the service tenancy 1919 of FIG. 19).

The control plane VCN 2116 can include a control plane DMZ tier 2120 (e.g., the control plane DMZ tier 1920 of FIG. 19) that can include load balancer (LB) subnet(s) 2122 (e.g., LB subnet(s) 1922 of FIG. 19), a control plane app tier 2124 (e.g., the control plane app tier 1924 of FIG. 19) that can include app subnet(s) 2126 (e.g., similar to app subnet(s) 1926 of FIG. 19), a control plane data tier 2128 (e.g., the control plane data tier 1928 of FIG. 19) that can include DB subnet(s) 2130. The LB subnet(s) 2122 contained in the control plane DMZ tier 2120 can be communicatively coupled to the app subnet(s) 2126 contained in the control plane app tier 2124 and to an Internet gateway 2134 (e.g., the Internet gateway 1934 of FIG. 19) that can be contained in the control plane VCN 2116, and the app subnet(s) 2126 can be communicatively coupled to the DB subnet(s) 2130 contained in the control plane data tier 2128 and to a service gateway 2136 (e.g., the service gateway of FIG. 19) and a network address translation (NAT) gateway 2138 (e.g., the NAT gateway 1938 of FIG. 19). The control plane VCN 2116 can include the service gateway 2136 and the NAT gateway 2138.

The data plane VCN 2118 can include a data plane app tier 2146 (e.g., the data plane app tier 1946 of FIG. 19), a data plane DMZ tier 2148 (e.g., the data plane DMZ tier 1948 of FIG. 19), and a data plane data tier 2150 (e.g., the data plane data tier 1950 of FIG. 19). The data plane DMZ tier 2148 can include LB subnet(s) 2122 that can be communicatively coupled to trusted app subnet(s) 2160 and untrusted app subnet(s) 2162 of the data plane app tier 2146 and the Internet gateway 2134 contained in the data plane VCN 2118. The trusted app subnet(s) 2160 can be communicatively coupled to the service gateway 2136 contained in the data plane VCN 2118, the NAT gateway 2138 contained in the data plane VCN 2118, and DB subnet(s) 2130 contained in the data plane data tier 2150. The untrusted app subnet(s) 2162 can be communicatively coupled to the service gateway 2136 contained in the data plane VCN 2118 and DB subnet(s) 2130 contained in the data plane data tier 2150. The data plane data tier 2150 can include DB subnet(s) 2130 that can be communicatively coupled to the service gateway 2136 contained in the data plane VCN 2118.

The untrusted app subnet(s) 2162 can include one or more primary VNICs 2164(1)-(N) that can be communicatively coupled to tenant virtual machines (VMs) 2166(1)-(N). Each tenant VM 2166(1)-(N) can be communicatively coupled to a respective app subnet 2167(1)-(N) that can be contained in respective container egress VCNs 2168(1)-(N) that can be contained in respective customer tenancies 2170(1)-(N). Respective secondary VNICs 2172(1)-(N) can facilitate communication between the untrusted app subnet(s) 2162 contained in the data plane VCN 2118 and the app subnet contained in the container egress VCNs 2168(1)-(N). Each container egress VCNs 2168(1)-(N) can include a NAT gateway 2138 that can be communicatively coupled to public Internet 2154 (e.g., public Internet 1954 of FIG. 19).

The Internet gateway 2134 contained in the control plane VCN 2116 and contained in the data plane VCN 2118 can be communicatively coupled to a metadata management service 2152 (e.g., the metadata management system 1952 of FIG. 19) that can be communicatively coupled to public Internet 2154. Public Internet 2154 can be communicatively coupled to the NAT gateway 2138 contained in the control plane VCN 2116 and contained in the data plane VCN 2118. The service gateway 2136 contained in the control plane VCN 2116 and contained in the data plane VCN 2118 can be communicatively coupled to cloud services 2156.

In some embodiments, the data plane VCN 2118 can be integrated with customer tenancies 2170. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 2146. Code to run the function may be executed in the VMs 2166(1)-(N), and the code may not be configured to run anywhere else on the data plane VCN 2118. Each VM 2166(1)-(N) may be connected to one customer tenancy 2170. Respective containers 2171(1)-(N) contained in the VMs 2166(1)-(N) may be configured to run the code. In this case, there can be a dual isolation (e.g., the containers 2171(1)-(N) running code, where the containers 2171(1)-(N) may be contained in at least the VM 2166(1)-(N) that are contained in the untrusted app subnet(s) 2162), which may help prevent incorrect or otherwise undesirable code from damaging the network of the IaaS provider or from damaging a network of a different customer. The containers 2171(1)-(N) may be communicatively coupled to the customer tenancy 2170 and may be configured to transmit or receive data from the customer tenancy 2170. The containers 2171(1)-(N) may not be configured to transmit or receive data from any other entity in the data plane VCN 2118. Upon completion of running the code, the IaaS provider may kill or otherwise dispose of the containers 2171(1)-(N).

In some embodiments, the trusted app subnet(s) 2160 may run code that may be owned or operated by the IaaS provider. In this embodiment, the trusted app subnet(s) 2160 may be communicatively coupled to the DB subnet(s) 2130 and be configured to execute CRUD operations in the DB subnet(s) 2130. The untrusted app subnet(s) 2162 may be communicatively coupled to the DB subnet(s) 2130, but in this embodiment, the untrusted app subnet(s) may be configured to execute read operations in the DB subnet(s) 2130. The containers 2171(1)-(N) that can be contained in the VM 2166(1)-(N) of each customer and that may run code from the customer may not be communicatively coupled with the DB subnet(s) 2130.

In other embodiments, the control plane VCN 2116 and the data plane VCN 2118 may not be directly communicatively coupled. In this embodiment, there may be no direct communication between the control plane VCN 2116 and the data plane VCN 2118. However, communication can occur indirectly through at least one method. An LPG 2110 may be established by the IaaS provider that can facilitate communication between the control plane VCN 2116 and the data plane VCN 2118. In another example, the control plane VCN 2116 or the data plane VCN 2118 can make a call to cloud services 2156 via the service gateway 2136. For example, a call to cloud services 2156 from the control plane VCN 2116 can include a request for a service that can communicate with the data plane VCN 2118.

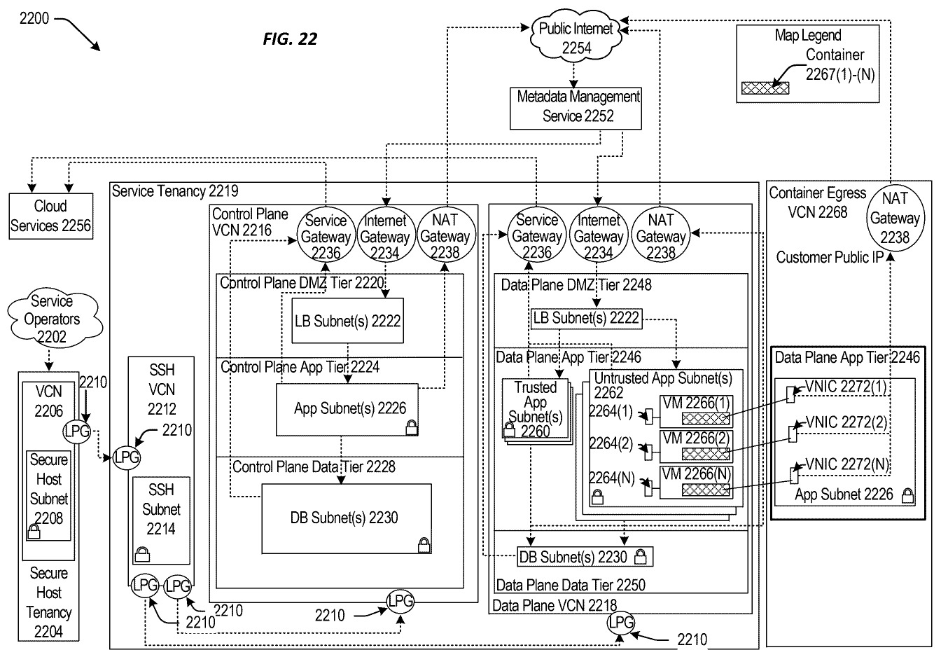

FIG. 22 is a block diagram 2200 illustrating another example pattern of an IaaS architecture, according to at least one embodiment. Service operators 2202 (e.g., service operators 1902 of FIG. 19) can be communicatively coupled to a secure host tenancy 2204 (e.g., the secure host tenancy 1904 of FIG. 19) that can include a virtual cloud network (VCN) 2206 (e.g., the VCN 1906 of FIG. 19) and a secure host subnet 2208 (e.g., the secure host subnet 1908 of FIG. 19). The VCN 2206 can include an LPG 2210 (e.g., the LPG 1910 of FIG. 19) that can be communicatively coupled to an SSH VCN 2212 (e.g., the SSH VCN 1912 of FIG. 19) via an LPG 2210 contained in the SSH VCN 2212. The SSH VCN 2212 can include an SSH subnet 2214 (e.g., the SSH subnet 1914 of FIG. 19), and the SSH VCN 2212 can be communicatively coupled to a control plane VCN 2216 (e.g., the control plane VCN 1916 of FIG. 19) via an LPG 2210 contained in the control plane VCN 2216 and to a data plane VCN 2218 (e.g., the data plane 1918 of FIG. 19) via an LPG 2210 contained in the data plane VCN 2218. The control plane VCN 2216 and the data plane VCN 2218 can be contained in a service tenancy 2219 (e.g., the service tenancy 1919 of FIG. 19).

The control plane VCN 2216 can include a control plane DMZ tier 2220 (e.g., the control plane DMZ tier 1920 of FIG. 19) that can include LB subnet(s) 2222 (e.g., LB subnet(s) 1922 of FIG. 19), a control plane app tier 2224 (e.g., the control plane app tier 1924 of FIG. 19) that can include app subnet(s) 2226 (e.g., app subnet(s) 1926 of FIG. 19), a control plane data tier 2228 (e.g., the control plane data tier 1928 of FIG. 19) that can include DB subnet(s) 2230 (e.g., DB subnet(s) 2130 of FIG. 21). The LB subnet(s) 2222 contained in the control plane DMZ tier 2220 can be communicatively coupled to the app subnet(s) 2226 contained in the control plane app tier 2224 and to an Internet gateway 2234 (e.g., the Internet gateway 1934 of FIG. 19) that can be contained in the control plane VCN 2216, and the app subnet(s) 2226 can be communicatively coupled to the DB subnet(s) 2230 contained in the control plane data tier 2228 and to a service gateway 2236 (e.g., the service gateway of FIG. 19) and a network address translation (NAT) gateway 2238 (e.g., the NAT gateway 1938 of FIG. 19). The control plane VCN 2216 can include the service gateway 2236 and the NAT gateway 2238.

The data plane VCN 2218 can include a data plane app tier 2246 (e.g., the data plane app tier 1946 of FIG. 19), a data plane DMZ tier 2248 (e.g., the data plane DMZ tier 1948 of FIG. 19), and a data plane data tier 2250 (e.g., the data plane data tier 1950 of FIG. 19). The data plane DMZ tier 2248 can include LB subnet(s) 2222 that can be communicatively coupled to trusted app subnet(s) 2260 (e.g., trusted app subnet(s) 2160 of FIG. 21) and untrusted app subnet(s) 2262 (e.g., untrusted app subnet(s) 2162 of FIG. 21) of the data plane app tier 2246 and the Internet gateway 2234 contained in the data plane VCN 2218. The trusted app subnet(s) 2260 can be communicatively coupled to the service gateway 2236 contained in the data plane VCN 2218, the NAT gateway 2238 contained in the data plane VCN 2218, and DB subnet(s) 2230 contained in the data plane data tier 2250. The untrusted app subnet(s) 2262 can be communicatively coupled to the service gateway 2236 contained in the data plane VCN 2218 and DB subnet(s) 2230 contained in the data plane data tier 2250. The data plane data tier 2250 can include DB subnet(s) 2230 that can be communicatively coupled to the service gateway 2236 contained in the data plane VCN 2218.

The untrusted app subnet(s) 2262 can include primary VNICs 2264(1)-(N) that can be communicatively coupled to tenant virtual machines (VMs) 2266(1)-(N) residing within the untrusted app subnet(s) 2262. Each tenant VM 2266(1)-(N) can run code in a respective container 2267(1)-(N), and be communicatively coupled to an app subnet 2226 that can be contained in a data plane app tier 2246 that can be contained in a container egress VCN 2268. Respective secondary VNICs 2272(1)-(N) can facilitate communication between the untrusted app subnet(s) 2262 contained in the data plane VCN 2218 and the app subnet contained in the container egress VCN 2268. The container egress VCN can include a NAT gateway 2238 that can be communicatively coupled to public Internet 2254 (e.g., public Internet 1954 of FIG. 19).

The Internet gateway 2234 contained in the control plane VCN 2216 and contained in the data plane VCN 2218 can be communicatively coupled to a metadata management service 2252 (e.g., the metadata management system 1952 of FIG. 19) that can be communicatively coupled to public Internet 2254. Public Internet 2254 can be communicatively coupled to the NAT gateway 2238 contained in the control plane VCN 2216 and contained in the data plane VCN 2218. The service gateway 2236 contained in the control plane VCN 2216 and contained in the data plane VCN 2218 can be communicatively coupled to cloud services 2256.

In some examples, the pattern illustrated by the architecture of block diagram 2200 of FIG. 22 may be considered an exception to the pattern illustrated by the architecture of block diagram 2100 of FIG. 21 and may be desirable for a customer of the IaaS provider if the IaaS provider cannot directly communicate with the customer (e.g., a disconnected region). The respective containers 2267(1)-(N) that are contained in the VMs 2266(1)-(N) for each customer can be accessed in real-time by the customer. The containers 2267(1)-(N) may be configured to make calls to respective secondary VNICs 2272(1)-(N) contained in app subnet(s) 2226 of the data plane app tier 2246 that can be contained in the container egress VCN 2268. The secondary VNICs 2272(1)-(N) can transmit the calls to the NAT gateway 2238 that may transmit the calls to public Internet 2254. In this example, the containers 2267(1)-(N) that can be accessed in real-time by the customer can be isolated from the control plane VCN 2216 and can be isolated from other entities contained in the data plane VCN 2218. The containers 2267(1)-(N) may also be isolated from resources from other customers.

In other examples, the customer can use the containers 2267(1)-(N) to call cloud services 2256. In this example, the customer may run code in the containers 2267(1)-(N) that requests a service from cloud services 2256. The containers 2267(1)-(N) can transmit this request to the secondary VNICs 2272(1)-(N) that can transmit the request to the NAT gateway that can transmit the request to public Internet 2254. Public Internet 2254 can transmit the request to LB subnet(s) 2222 contained in the control plane VCN 2216 via the Internet gateway 2234. In response to determining the request is valid, the LB subnet(s) can transmit the request to app subnet(s) 2226 that can transmit the request to cloud services 2256 via the service gateway 2236.

It should be appreciated that IaaS architectures 1900, 2000, 2100, 2200 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

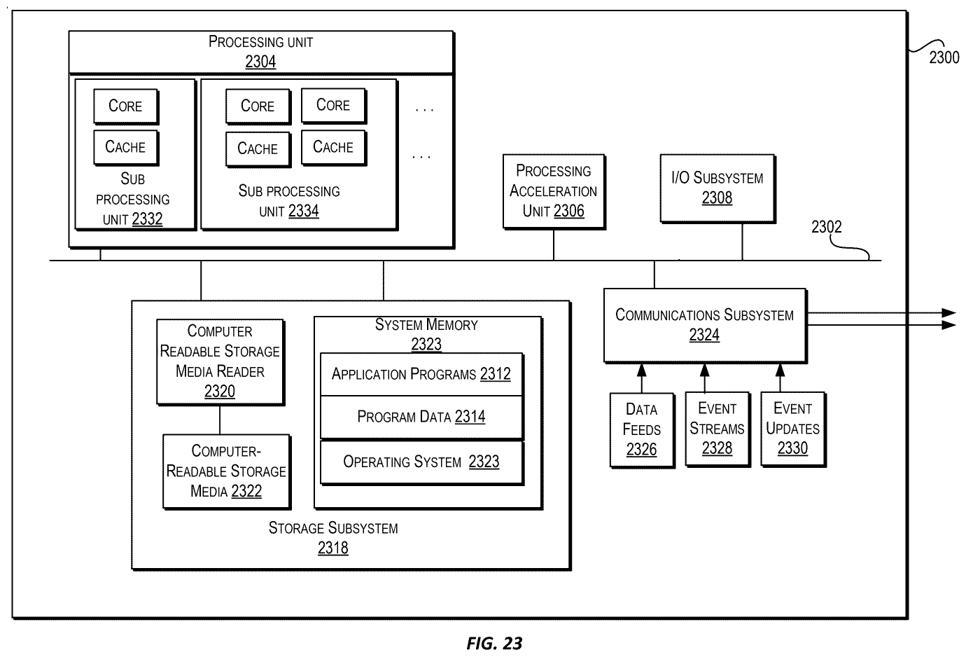

FIG. 23 illustrates an example computer system 2300, in which various embodiments may be implemented. The system 2300 may be used to implement any of the computer systems described above. As shown in the figure, computer system 2300 includes a processing unit 2304 that communicates with a number of peripheral subsystems via a bus subsystem 2302. These peripheral subsystems may include a processing acceleration unit 2306, an I/O subsystem 2308, a storage subsystem 2318 and a communications subsystem 2324. Storage subsystem 2318 includes tangible computer-readable storage media 2322 and a system memory 2310.

Bus subsystem 2302 provides a mechanism for letting the various components and subsystems of computer system 2300 communicate with each other as intended. Although bus subsystem 2302 is shown schematically as a single bus, alternative embodiments of the bus subsystem may utilize multiple buses. Bus subsystem 2302 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 2304, which can be implemented as one or more integrated circuits (e.g., a conventional microprocessor or microcontroller), controls the operation of computer system 2300. One or more processors may be included in processing unit 2304. These processors may include single core or multicore processors. In certain embodiments, processing unit 2304 may be implemented as one or more independent processing units 2332 and/or 2334 with single or multicore processors included in each processing unit. In other embodiments, processing unit 2304 may also be implemented as a quad-core processing unit formed by integrating two dual-core processors into a single chip.

In various embodiments, processing unit 2304 can execute a variety of programs in response to program code and can maintain multiple concurrently executing programs or processes. At any given time, some or all of the program code to be executed can be resident in processor(s) 2304 and/or in storage subsystem 2318. Through suitable programming, processor(s) 2304 can provide various functionalities described above. Computer system 2300 may additionally include a processing acceleration unit 2306, which can include a digital signal processor (DSP), a special-purpose processor, and/or the like.

I/O subsystem 2308 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 2300 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 2300 may comprise a storage subsystem 2318 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 2304 provide the functionality described above. Storage subsystem 2318 may also provide a repository for storing data used in accordance with the present disclosure.

As depicted in the example in FIG. 23, storage subsystem 2318 can include various components including a system memory 2310, computer-readable storage media 2322, and a computer readable storage media reader 2320. System memory 2310 may store program instructions that are loadable and executable by processing unit 2304. System memory 2310 may also store data that is used during the execution of the instructions and/or data that is generated during the execution of the program instructions. Various different kinds of programs may be loaded into system memory 2310 including but not limited to client applications, Web browsers, mid-tier applications, relational database management systems (RDBMS), virtual machines, containers, etc.

System memory 2310 may also store an operating system 2316. Examples of operating system 2316 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 2300 executes one or more virtual machines, the virtual machines along with their guest operating systems (GOSs) may be loaded into system memory 2310 and executed by one or more processors or cores of processing unit 2304.

System memory 2310 can come in different configurations depending upon the type of computer system 2300. For example, system memory 2310 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 2310 may include a basic input/output system (BIOS) containing basic routines that help to transfer information between elements within computer system 2300, such as during start-up.

Computer-readable storage media 2322 may represent remote, local, fixed, and/or removable storage devices plus storage media for temporarily and/or more permanently containing, storing, computer-readable information for use by computer system 2300 including instructions executable by processing unit 2304 of computer system 2300.

Computer-readable storage media 2322 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 2322 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 2322 may include, but is not limited to, Zip® drives, flash memory cards, universal serial bus (USB) flash drives, secure digital (SD) cards, DVD disks, digital video tape, and the like. Computer-readable storage media 2322 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 2300.

Machine-readable instructions executable by one or more processors or cores of processing unit 2304 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 2324 provides an interface to other computer systems and networks. Communications subsystem 2324 serves as an interface for receiving data from and transmitting data to other systems from computer system 2300. For example, communications subsystem 2324 may enable computer system 2300 to connect to one or more devices via the Internet. In some embodiments communications subsystem 2324 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 2324 can provide wired network connectivity (e.g., Ethernet) in addition to or instead of a wireless interface.

In some embodiments, communications subsystem 2324 may also receive input communication in the form of structured and/or unstructured data feeds 2326, event streams 2328, event updates 2330, and the like on behalf of one or more users who may use computer system 2300.

By way of example, communications subsystem 2324 may be configured to receive data feeds 2326 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 2324 may also be configured to receive data in the form of continuous data streams, which may include event streams 2328 of real-time events and/or event updates 2330, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 2324 may also be configured to output the structured and/or unstructured data feeds 2326, event streams 2328, event updates 2330, and the like to one or more databases that may be in communication with one or more streaming data source computers coupled to computer system 2300.

Computer system 2300 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 2300 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
obtaining, in response to a clinical query associated with an anticipated or ongoing patient encounter, an intermediate representation of patient-specific data, the intermediate representation comprising semantic objects extracted from one or more data sources, the semantic objects comprising a condition and a medication associated with a patient, and the intermediate representation further representing a reason for visit and a chief complaint for the encounter;
processing the intermediate representation via a transform layer comprising multiple filtering modules to extract condition-related and medication-related information relevant to the encounter;
processing outputs of the transform layer via an enrichment layer to further filter and contextualize subsets of the condition-related and medication-related information, wherein processing the outputs comprises extracting a subset of medications known to have therapeutic or adverse effects with respect to filtered conditions and extracting a subset of conditions affiliated with the subset of medications;
within the enrichment layer, applying one or more filtering techniques including a knowledge-graph-based filtering technique, wherein the knowledge-graph-based filtering technique prioritizes and retains entities according to a relevancy score; and
generating, based on outputs of the transform layer and the enrichment layer, a clinical summary for the patient, the clinical summary comprising a narrative summary generated using natural language processing and a structured summary comprising structured facts derived from the intermediate representation.

2. The method of claim 1, wherein obtaining the intermediate representation comprises:
receiving the clinical query;
identifying, in the clinical query, clinical entities including at least medication entities and condition entities; and
codifying the clinical entities using one or more coding systems to form a data structure compliant with an electronic healthcare information exchange standard.

3. The method of claim 1, further comprising:
generating, using one or more knowledge sources and one or more large language models, a semantic knowledge graph representing clinical entities and typed relationships among the clinical entities;
utilizing the semantic knowledge graph to prioritize and filter coded entities identified from the clinical query and the intermediate representation to produce link data comprising at least a prioritized condition list, a prioritized medication list, and relationships among conditions and medications; and
supplying the link data to the enrichment layer to guide selection of clinically relevant entities for inclusion in the clinical summary.

4. The method of claim 3, wherein generating the semantic knowledge graph comprises:
executing a multi-stage pipeline including a discovery stage that applies a large language model to propose candidate relationships between clinical entities for which discretely documented links are absent in available knowledge sources;
validating the candidate relationships; and
persisting, for each candidate relationship of the candidate relationships, metadata comprising at least a source attribution that identifies a large language model, and a relationship type.

5. The method of claim 3, wherein the link data comprises relationship sets including:
relationships that link a reason for visit or a chief complaint to candidate conditions relevant to encounter context;
relationships that link the reason for visit or the chief complaint directly to candidate medications relevant to context of the encounter;
relationships that link candidate conditions to candidate medications using relationship types; and
relationships obtained indirectly by first identifying candidate conditions and then linking the candidate conditions to candidate medications.

6. The method of claim 3, wherein generating the clinical summary comprises:
populating a summary data structure with the link data such that a narrative portion of the clinical summary describes, in natural language, a context of the encounter and clinically prioritized entities and a structured portion of the clinical summary presents structured facts selected from the prioritized condition list, the prioritized medication list, and links that explain a rationale for prioritization and filtering.

7. The method of claim 3, further comprising:
maintaining the semantic knowledge graph, wherein maintaining the semantic knowledge graph comprises:
normalizing source data to ontology-aligned representations;
applying constraint and validation artifacts to detect incompleteness or inconsistency and reclassifying nodes or edges when ontologies or value sets are updated; and
incrementally enriching the semantic knowledge graph over time by ingesting outputs of a large language model.

8. A system comprising:
one or more processors; and
one or more computer-readable media storing instructions which, when executed by the one or more processors, cause the system to perform operations comprising:
obtaining, in response to a clinical query associated with an anticipated or ongoing patient encounter, an intermediate representation of patient-specific data, the intermediate representation comprising semantic objects extracted from one or more data sources, the semantic objects comprising a condition and a medication associated with a patient, and the intermediate representation further representing a reason for visit and a chief complaint for the encounter;
processing the intermediate representation via a transform layer comprising multiple filtering modules to extract condition-related and medication-related information relevant to the encounter;
processing outputs of the transform layer via an enrichment layer to further filter and contextualize subsets of the condition-related and medication-related information, wherein processing the outputs comprises extracting a subset of medications known to have therapeutic or adverse effects with respect to filtered conditions and extracting a subset of conditions affiliated with the subset of medications;
within the enrichment layer, applying one or more filtering techniques including a knowledge-graph-based filtering technique, wherein the knowledge-graph-based filtering technique prioritizes and retains entities according to a relevancy score; and
generating, based on outputs of the transform layer and the enrichment layer, a clinical summary for the patient, the clinical summary comprising a narrative summary generated using natural language processing and a structured summary comprising structured facts derived from the intermediate representation.

9. The system of claim 8, wherein obtaining the intermediate representation comprises:
receiving the clinical query;
identifying, in the clinical query, clinical entities including at least medication entities and condition entities; and
codifying the clinical entities using one or more coding systems to form a data structure compliant with an electronic healthcare information exchange standard.

10. The system of claim 8, the operations further comprising:
generating, using one or more knowledge sources and one or more large language models, a semantic knowledge graph representing clinical entities and typed relationships among the clinical entities;
utilizing the semantic knowledge graph to prioritize and filter coded entities identified from the clinical query and the intermediate representation to produce link data comprising at least a prioritized condition list, a prioritized medication list, and relationships among conditions and medications; and
supplying the link data to the enrichment layer to guide selection of clinically relevant entities for inclusion in the clinical summary.

11. The system of claim 10, wherein generating and maintain the semantic knowledge graph comprises:
executing a multi-stage pipeline including a discovery stage that applies a large language model to propose candidate relationships between clinical entities for which discretely documented links are absent in available knowledge sources;
validating the candidate relationships; and
persisting, for each candidate relationship of the candidate relationships, metadata comprising at least a source attribution that identifies a large language model, and a relationship type.

12. The system of claim 10, wherein the link data comprises relationship sets including:
relationships that link a reason for visit or a chief complaint to candidate conditions relevant to encounter context;
relationships that link the reason for visit or the chief complaint directly to candidate medications relevant to context of the encounter;
relationships that link candidate conditions to candidate medications using relationship types; and
relationships obtained indirectly by first identifying candidate conditions and then linking the candidate conditions to candidate medications.

13. The system of claim 10, wherein generating the clinical summary comprises:
populating a summary data structure with the link data such that a narrative portion of the clinical summary describes, in natural language, a context of the encounter and clinically prioritized entities and a structured portion of the clinical summary presents structured facts selected from the prioritized condition list, the prioritized medication list, and links that explain a rationale for prioritization and filtering.

14. The system of claim 11, the operations further comprising:
maintaining the semantic knowledge graph, wherein maintaining the semantic knowledge graph comprises:
normalizing source data to ontology-aligned representations;
applying constraint and validation artifacts to detect incompleteness or inconsistency and reclassifying nodes or edges when ontologies or value sets are updated; and
incrementally enriching the semantic knowledge graph over time by ingesting outputs of a large language model.

15. One or more non-transitory computer-readable media storing instructions which, when executed by one or more processors, cause a system to perform operations comprising:
obtaining, in response to a clinical query associated with an anticipated or ongoing patient encounter, an intermediate representation of patient-specific data, the intermediate representation comprising semantic objects extracted from one or more data sources, the semantic objects comprising a condition and a medication associated with a patient, and the intermediate representation further representing a reason for visit and a chief complaint for the encounter;
processing the intermediate representation via a transform layer comprising multiple filtering modules to extract condition-related and medication-related information relevant to the encounter;
processing outputs of the transform layer via an enrichment layer to further filter and contextualize subsets of the condition-related and medication-related information, wherein processing the outputs comprises extracting a subset of medications known to have therapeutic or adverse effects with respect to filtered conditions and extracting a subset of conditions affiliated with the subset of medications;
within the enrichment layer, applying one or more filtering techniques including a knowledge-graph-based filtering technique, wherein the knowledge-graph-based filtering technique prioritizes and retains entities according to a relevancy score; and
generating, based on outputs of the transform layer and the enrichment layer, a clinical summary for the patient, the clinical summary comprising a narrative summary generated using natural language processing and a structured summary comprising structured facts derived from the intermediate representation.

16. The one or more non-transitory computer-readable media of claim 15, wherein obtaining the intermediate representation comprises:
receiving the clinical query;
identifying, in the clinical query, clinical entities including at least medication entities and condition entities; and
codifying the clinical entities using one or more coding systems to form a data structure compliant with an electronic healthcare information exchange standard.

17. The one or more non-transitory computer-readable media of claim 15, the operations further comprising:
generating, using one or more knowledge sources and one or more large language models, a semantic knowledge graph representing clinical entities and typed relationships among the clinical entities;
utilizing the semantic knowledge graph to prioritize and filter coded entities identified from the clinical query and the intermediate representation to produce link data comprising at least a prioritized condition list, a prioritized medication list, and relationships among conditions and medications; and
supplying the link data to the enrichment layer to guide selection of clinically relevant entities for inclusion in the clinical summary.

18. The one or more non-transitory computer-readable media of claim 17, wherein generating the semantic knowledge graph comprises:
executing a multi-stage pipeline including a discovery stage that applies a large language model to propose candidate relationships between clinical entities for which discretely documented links are absent in available knowledge sources;
validating the candidate relationships; and
persisting, for each candidate relationship of the candidate relationships, metadata comprising at least a source attribution that identifies a large language model, and a relationship type.

19. The one or more non-transitory computer-readable media of claim 17, wherein the link data comprises relationship sets including:
relationships that link a reason for visit or a chief complaint to candidate conditions relevant to encounter context;
relationships that link the reason for visit or the chief complaint directly to candidate medications relevant to context of the encounter;
relationships that link candidate conditions to candidate medications using relationship types; and
relationships obtained indirectly by first identifying candidate conditions and then linking the candidate conditions to candidate medications.

20. The one or more non-transitory computer-readable media of claim 17, wherein generating the clinical summary comprises:
populating a summary data structure with the link data such that a narrative portion of the clinical summary describes, in natural language, a context of the encounter and clinically prioritized entities and a structured portion of the clinical summary presents structured facts selected from the prioritized condition list, the prioritized medication list, and links that explain a rationale for prioritization and filtering.
